(19) United States
(12) Patent Application Publication
(10) Pub. No.: US 2026/0196315 A1
(43) Pub. Date: Jul. 9, 2026

(54) ENCOUNTER RECORD AND PATIENT CHART SYNCHRONIZATION SYSTEM FOR MEDICAL RECORDS

(71) Applicant: Oak Street Health, Chicago, IL (US)

(72) Inventors: Geoff Neihaus, Woonsocket, RI (US); Alexander Hubitski, Woonsocket, RI (US); Hayk Ghukasyan, Woonsocket, RI (US); Lotus Verano Lindez, Orlando, FL (US); Leigha Grimm, Palatine, IL (US); Alison Dougherty, Woonsocket, RI (US); Amy Ingebretson, Apex, NC (US); Sydney Simon, Brooklyn, NY (US); William Chung, Tampa, FL (US); Emily DeMichele, Encinitas, CA (US); Kristin Roy, Amherst, MA (US); Mildred Bley, Franklin, WI (US)

(73) Assignee: Oak Street Health, Chicago, IL (US)

(21) Appl. No.: 19/011,525

(22) Filed: Jan. 6, 2025

(51) Int. Cl. G16H 10/60; G16H 80/00

(52) U.S. Cl. CPC G16H 10/60; G16H 80/00

(57) ABSTRACT

Systems and methods for synchronizing notes for a visit of a patient with a healthcare provider with a stored record for the patient. The method may include providing, by one or more processing circuits, a first user interface. The first user interface may capture information about the patient or the visit. The method may include obtaining an edit to the information from a first user. The method may include detecting a trigger event. In response, the processing circuits may finalize the edit from the first user. The method may include saving the edit to the stored record for the patient. The method may include pushing an update to at least one of the first user interface for the first user or a second user interface for a second user. Pushing the update may cause the first user interface or the second user interface to be updated based on the edit.

### FIELD

This present disclosure relates generally to systems and methods for managing records of a visit to a medical provider. Various examples relate to multi-tenant access for a medical record system (e.g., facilitating multiple users editing the medical records) and/or synchronizing records relating to the visit across multiple types of records and/or multiple systems.

### BACKGROUND

When a patient visits a medical provider, they may interact with a number of different people. Various people may make edits to records relating to the visit before, during, and/or after the visit. It would be beneficial to have a system that manages the records to help avoid conflicts from edits by different users and ensure records are properly updated and synchronized.

### SUMMARY

At least one example is directed to a method for capturing records for a patient of a visit with a healthcare provider from a plurality of users during a same timeframe. The method may include providing, via one or more processing circuits, a user interface comprising a plurality of sections. The plurality of sections may capture a plurality of different pieces of information regarding at least one of the patient or the visit. The method may include detecting, using the one or more processing circuits, that a first user of the plurality of users is editing a first section of the plurality of sections. The method may include locking, using the one or more processing circuits, responsive to detecting that the first user is editing the first section, the first section to editing by a second user and one or more third users of the plurality of users. The method may include detecting, using the one or more processing circuits, that the first user has finished editing the first section. The method may include, responsive to detecting that the first user has finished editing the first section, unlocking, using the one or more processing circuits, the first section for editing by the second user and the one or more third users.

In some examples, the method further includes detecting, using the one or more processing circuits, that the second user is editing a second section of the plurality of sections. The method may include locking, using the one or more processing circuits, responsive to detecting that the second user is editing the second section, the second section to editing by the first user and the one or more third users. The method may include detecting, using the one or more processing circuits, that the second user has finished editing the second section. The method may include unlocking, using the one or more processing circuits, responsive to detecting that the second user has finished editing the second section, the second section for editing by the first user and the one or more third users.

In some examples, locking the second section to editing by the first user and the one or more third users occurs during the timeframe that the first section is locked to editing by the second user and the one or more third users. The method may include permitting, using the one or more processing circuits, editing to a third section by the one or more third users while the first section and the second section are locked to editing by the one or more third users.

In some examples, the method further includes providing, by the one or more processing circuits responsive to locking the first section, identity information to the user interface for the second user and the one or more third users. The identity information may indicate an identity of the first user editing the first section.

In some examples, the method further includes providing, by the one or more processing circuits responsive to locking the first section, an indication to the second user and the one or more third users that are viewing the first section. The indication may indicate that the first section is locked.

In some examples, the method further includes providing, by the one or more processing circuits responsive to unlocking the first section, an indication to the second user and the one or more third users that are viewing the first section. The indication may indicate that the first section has been unlocked.

In some examples, the user interface may include at least two portions provided on a same display interface. The method may further include displaying, via the user interface, a first portion of the at least two portions. The first portion may include a section configured to capture the records for the visit. The first portion may include the first section edited by the first user. The method may further include displaying, via the user interface, a second portion of the at least two portions. The second portion may include a condensed health record of the patient.

In some examples, the method may further include displaying, via the user interface, a third portion of the at least two portions. The third portion may include a selection menu configured to receive a request from the second user or the one or more third users to view a desired section of the plurality of sections. The method may further include displaying, via the user interface, the desired section of the plurality of sections on the first portion of the user interface.

In some examples, the method may further include receiving, by the one or more processing circuits, one or more edits to the first section from the first user. The method may further include storing, by the one or more processing circuits, the one or more edits to the first section. The method may further include transmitting, by the one or more processing circuits, at least a portion of the records, including the edits to the first section, to at least a portion of a condensed healthcare record of the patient.

In some examples, the method may further include receiving, by the one or more processing circuits, one or more edits to the first section from the first user. The method may further include storing, by the one or more processing circuits, the one or more edits to the first section. The method may further include updating, by the one or more processing circuits, a portion of the user interface including the first section, to display the records associated with the first section, including the edits from the first user, to the second user and the one or more third users.

In some examples, the method may further include receiving, by the one or more processing circuits, one or more edits to the first section from the first user. The method may further include storing, by the one or more processing circuits, the one or more edits to the first section. The method may further include updating, by the one or more processing circuits, a portion of the user interface including the first section, to display the one or more records of the first section, including the edits from the first user, to the second user and the one or more third users. The portion of the user interface including the first section may be updated within a threshold amount of time from when the first user edits the first section.

In some examples, the method further includes receiving, by the one or more processing circuits, one or more records of the first section from the first user. The method may further include storing, by the one or more processing circuits, the one or more records of the first section. The method may further include receiving, by the one or more processing circuits, a validation of the one or more records of the first section from the first user. The method may further include responsive to receiving the validation, updating, by the one or more processing circuits, a portion of the user interface including the first section, to display the one or more records of the first section to the second user and the one or more third users. The method may further include updating, by the one or more processing circuits, a second portion of the user interface including a condensed health record of the patient. Updating the second portion may include updating the condensed health record with information from the one or more records of the first section. The method may further include displaying, by the one or more processing circuits, the condensed health record, including the edits of the first section, to the second user and the one or more third users.

In some examples, the method further includes receiving, by the one or more processing circuits, an indication that the first user has been inactive for a predefined time limit. The method may further include unlocking, by the one or more processing circuits responsive to the indication, the first section for editing by the second user and the one or more third users.

In some aspects, the techniques described herein relate to a non-transitory computer-readable storage media, wherein the instructions further cause the one or more processors to perform operations including: receiving, by the one or more processors, one or more edits to at least one of the first section from the first user or the second section from the second user; storing, by the one or more processors, the one or more edits; displaying, via the user interface, a first portion of the at least two portions, wherein the first portion includes a section configured to capture records for the visit, the first portion including at least one of the first section edited by the first user or the second section edited by the second user; transmitting, by the one or more processors, at least a portion of the records, including the one or more edits, to at least a portion of a condensed healthcare record of the patient; and displaying, via the user interface, a second portion of the at least two portions, wherein the second portion includes a condensed health record of the patient.

At least one example is directed to a system to capture records for a visit of a patient with a healthcare provider from a plurality of users during a same timeframe. The system may include one or more processing circuits configured to provide a user interface comprising a plurality of sections. The plurality of sections may capture a plurality of different pieces of information regarding at least one of the patient or the meeting. The one or more processing circuits may detect that a first user of the plurality of users is editing a first section of the plurality of sections. The one or more processing circuits may, responsive to detecting that the first user is editing the first section, lock the first section to editing by a second user and one or more third users of the plurality of users. The one or more processing circuits may detect that the first user has finished editing the first section. The one or more processing circuits can, responsive to detecting that the first user has finished editing the first section, unlock the first section for editing by the second user and the one or more third users.

In some examples, the one or more processing circuits are further configured to detect that the second user is editing a second section of the plurality of sections. The one or more processing circuits may, responsive to detecting that the second user is editing the second section, lock the second section to editing by the first user and the one or more third users. The one or more processing circuits may detect that the second user has finished editing the second section. The one or more processing circuits may, responsive to detecting that the second user has finished editing the second section, unlock the second section for editing by the first user and the one or more third users.

In some examples, the one or more processing circuits are further configured to provide an indication, on the user interface, to the second user and the one or more third users that are viewing the first section. The indication may indicate that the first section is either locked or has been unlocked.

In some examples, the one or more processing circuits are further configured to receive one or more edits to the first section from the first user. The one or more processing circuits may store the one or more edits to the first section. The one or more processing circuits may update a portion of the user interface including the first section to display the records associated with the first section, including the edits from the first user, to the second user and the one or more third users.

In some examples, the one or more processing circuits are further configured to receive one or more records, including one or more edits to the first section, from the first user. The one or more processing circuits may store the one or more records associated with the first section. The one or more processing circuits may receive a validation of the one or more records of the first section from the first user. The one or more processing circuits may, responsive to receiving the validation, update a portion of the user interface including the first section, to display the one or more records of the first section to the second user and the one or more third users.

At least one example is directed to one or more non-transitory computer-readable storage media. The media may have instructions thereon that, when executed by one or more processors, cause the one or more processors to perform operations including providing a user interface. The user interface may include a plurality of sections capturing a plurality of different pieces of information regarding a visit of a patient with a healthcare provider. The operations may include detecting that a first user of a plurality of users is editing a first section of the plurality of sections. The operations may include, responsive to detecting that the first user is editing the first section, locking the first section to editing by a second user and one or more third users of the plurality of users. The operations may include permitting edits to a second section of the plurality of sections by the second user while the first section is locked by the first user. The operations may include detecting that the first user has finished editing the first section. The operations may include, responsive to detecting that the first user has finished editing the first section, unlocking the first section for editing by the second user and the one or more third users.

In some examples, the operations further include receiving, by the one or more processors, one or more edits to at least one of the first section from the first user or the second section from the second user. The operations may include storing, by the one or more processors, the one or more edits. The operations may include displaying, via the user interface, a first portion of the at least two portions. The first portion may include a section configured to capture records for the visit. The first portion may include at least one of the first section edited by the first user or the second section edited by the second user. The operations may include transmitting, by the one or more processors, at least a portion of the records, including the one or more edits, to at least a portion of a condensed healthcare record of the patient. The operations may include displaying, via the user interface, a second portion of the at least two portions. The second portion may include a condensed health record of the patient.

At least one example is directed to a method for synchronizing notes for a visit of a patient with a healthcare provider with a stored record for the method. The method may include providing, by one or more processing circuits, a first user interface. The first user interface may capture information regarding at least one of the patient or the visit. The method may include obtaining, via the first user interface, an edit to the information from a first user. The method may include detecting a trigger event. Responsive to detecting the trigger event, the one or more processing circuits may finalize the edit to the information from the first user. The method may include saving, by the one or more processing circuits, responsive to finalizing the edit, the edit to the stored record for the patient. The method may include pushing, responsive to finalizing the edit, by the one or more processing circuits, an update based on the edit to at least one of the first user interface being viewed by the first user or a second user interface being viewed by a second user. Pushing the update may cause a portion of the at least one of the first user interface or the second user interface associated with the stored record to be updated based on the edit.

In some examples, at least one of the first user interface or the second user interface may include a plurality of sections. The plurality of sections may capture a plurality of different pieces of information regarding at least one of the patient or the visit. The plurality of sections may include at least one of: a visit purpose section, a vitals section, a screenings section, a medications section, a point of care section, an immunizations section, a history section, an allergy section, demographics section, an assessment section, a care plan section, a physical exam section, or a subjective notes section.

In some examples, at least one of the first user interface or the second user interface may include a first portion and a second portion provided within a same display screen. The method may further include receiving the edit to the information, by the one or more processing circuits, in the first portion of the first user interface. The method may further include pushing the update based on the edit to the second portion of the first user interface.

In some examples, the second portion may be a condensed health record of the patient. Pushing the update based on the edit may cause at least a portion of the condensed health record of the first user interface and the second user interface to update based on the edit.

In some examples, pushing the update may be done within a threshold time after the edit to the information is received.

In some examples, the edit to the information may be obtained from the first user interface of the first user and the update may be pushed to the second user interface of the second user.

In some examples, the trigger event may include at least one of an information approval event or an editing timeout event.

In some examples, at least one of the first user interface or the second user interface may include a plurality of sections capturing a plurality of different pieces of information regarding at least one of the patient or the visit. The method may further include detecting, using the one or more processing circuits, that the first user is editing a first section of the plurality of sections. The method may further include responsive to detecting that the first user is editing the first section, locking, using the one or more processing circuits, the first section to editing by other users of a plurality of users.

In some examples, the method further includes detecting, using the one or more processing circuits, that the first user has finished editing the first section. The method may further include responsive to detecting that the first user has finished editing the first section, unlocking, using the one or more processing circuits, the first section for editing by the second user.

In some examples, at least one of the first user interface or the second user interface includes a plurality of sections capturing a plurality of different pieces of information regarding at least one of the patient or the visit. The method may further include obtaining, via the first user interface, the edit to the information from the first user editing a first section of the plurality of sections. The method may further include updating, via the first user interface, other sections of the plurality of sections with at least a portion of the edit to the information from the first user.

In some examples, the method further includes capturing, via the one or more processing circuits, the edit to the information. The method may further include storing, via a database, the edit to the information. The method may further include responsive to detecting the trigger event, saving the edit to the stored record for the patient.

In some examples, at least one of the first user interface or the second user interface includes a plurality of sections capturing a plurality of different pieces of information regarding at least one of the patient or the visit. The method may further include displaying, via the first user interface, a first portion of the plurality of sections to the first user. The first portion of the plurality of sections may be based on a first role assigned to the first user. The method may further include displaying, via the second user interface, a second portion of the plurality of sections to the second user. The second portion of the plurality of sections may be based on a second role assigned to the second user. The second portion may include different information than the first portion and the second role may be different than the first role.

In some examples, the method may further include determining, by the one or more processing circuits, a plurality of at least one of health screening recommendations or examination recommendations related to at least one of the patient or the visit. The method may further include providing, via the one or more processing circuits, screening information including the plurality of the at least one of the health screening recommendations or the examination recommendations on the first user interface.

In some examples, the method may include providing, via the one or more processing circuits, statement information on the first user interface. The statement information may have a plurality of statement codes associated with at least one of the patient or the visit. The plurality of statement codes may be determined, by the one or more processing circuits, based on the information.

At least one example is directed to a system to synchronize notes for a visit of a patient with a healthcare provider with a stored record for the patient. The system may include one or more processing circuits configured to provide a first user interface. The first user interface may capture information regarding at least one of the patient or the visit. The one or more processing circuits may obtain an edit to the information from a first user. The one or more processing circuits may detect a trigger event. Responsive to detecting the trigger event, the one or more processing circuits may finalize the edit to the information from the first user. Responsive to finalizing the edit, the one or more processing circuits may finalize the edit to the information from the first user. Responsive to finalizing the edit, the one or more processing circuits may push an update based on the edit to at least one of the first user interface being viewed by the first user or a second user interface being viewed by a second user. Pushing the update may include causing a portion of at least one of the first user interface or the second user interface associated with the stored record to be updated based on the edit.

In some examples, at least one of the first user interface or the second user interface includes a plurality of sections capturing a plurality of different pieces of information regarding at least one of the patient or the visit. The one or more processing circuits may detect that the first user is editing a first section of the plurality of sections. The one or more processing circuits may, responsive to detecting that the first user is editing the first section, lock the first section to editing by other users of a plurality of users.

In some examples, at least one of the first user interface or the second user interface includes a plurality of sections capturing a plurality of different pieces of information regarding at least one of the patient or the visit. The plurality of sections may include at least one of: a visit purpose section, a vitals section, a screenings section, a medications section, a point of care section, an immunizations section, a history section, an allergy section, and a demographics section, an assessment section, a care plan section, a physical exam section, or a subjective notes section.

In some examples, the one or more processing circuits are further configured to capture the edit to the information. The one or more processing circuits are further configured to store, via a database, the edit to the information, The one or more processing circuits are further configured to, responsive to detecting the trigger event, save the edit to the stored record for the patient.

In some examples, at least one of the first user interface or the second user interface include a first portion and a second portion within a same display screen. The one or more processing circuits may be further configured to receive the edit to the information in the first portion of the first user interface. The one or more processing circuits may be further configured to push the update based on the edit to the information to the second portion of the first user interface. The second portion may be a summarized health record of the patient.

At least one example is directed to one or more non-transitory computer-readable storage media. The media can have instructions thereon that, when executed by one or more processors, cause the one or more processors to perform operations including providing a user interface. The user interface can include information regarding a visit of a patient with a healthcare provider. The operations can include obtaining an edit to the information from a first user. The operations can include detecting a trigger event. Responsive to detecting the trigger event, the operations can include finalizing the edit to the information from the first user. The operations can include, responsive to finalizing the edit, saving the edit to a stored record for the patient. The operations can include, responsive to finalizing the edit, pushing an update based on the edit. The update may be pushed to at least one of the first user interface being viewed by the first user or a second user interface being viewed by a second user. Pushing the update may cause a portion of at least one of the first user interface or the second user interface associated with the stored record to be updated based on the edit.

All examples and features mentioned above can be combined in any technically possible way.

These and other aspects and implementations are discussed in detail below. The foregoing information and the following detailed description include illustrative examples of various aspects and implementations, and provide an overview or framework for understanding the nature and character of the claimed aspects and implementations. The drawings provide illustration and a further understanding of the various aspects and implementations, and are incorporated in and constitute a part of this specification. The foregoing information and the following detailed description and drawings include illustrative examples and should not be considered as limiting.

### BRIEF DESCRIPTION OF THE DRAWINGS

The accompanying drawings are not intended to be drawn to scale. Like reference numbers and designations in the various drawings indicate like elements. For purposes of clarity, not every component may be labeled in every drawing. In the drawings:

FIG. 1 is an example system for capturing records from a plurality of users.

FIG. 2 is a flow diagram of a process for capturing records with the system of FIG. 1, according to some examples.

FIG. 3 is a flow diagram of a locking process, according to some examples.

FIG. 4 is a flow diagram of another locking process, according to some examples.

FIG. 5 is a flow diagram of another locking process, according to some examples.

FIG. 6 is a flow diagram of another locking process, according to some examples.

FIG. 7 is a flow diagram of another locking process, according to some examples.

FIG. 8 is a user interface to display and capture records, according to some examples.

FIG. 9 is another user interface to display and capture records, according to some examples.

FIG. 10 is another user interface to display and capture records, according to some examples.

FIG. 11 is another user interface to display and capture records, according to some examples.

FIG. 12 is another user interface to display and capture records, according to some examples.

FIG. 13 is another user interface to display and capture records, according to some examples.

FIG. 14 is another user interface to display and capture records, according to some examples.

FIG. 15 is another user interface to display and capture records, according to some examples.

FIG. 16 is another user interface to display and capture records, according to some examples.

FIG. 17 is another user interface to display and capture records, according to some examples.

FIG. 18 is another user interface to display and capture records, according to some examples.

FIG. 19 is another user interface to display and capture records, according to some examples.

FIG. 20 is another user interface to display and capture records, according to some examples.

FIG. 21 is another user interface to display and capture records, according to some examples.

FIG. 22 is another user interface to display and capture records, according to some examples.

FIG. 23 is another user interface to display and capture records, according to some examples.

FIG. 24A is another user interface to display and capture records, according to some examples.

FIG. 24B is another user interface to display and capture records, according to some examples.

FIG. 25 is a flow diagram of a process of locking sections of a patient record for editing, according to some examples.

FIG. 26 is a flow diagram of a process of synchronizing records, according to some examples.

### DETAILED DESCRIPTION

Following below are more detailed descriptions of various concepts related to, and implementations of, methods, apparatuses, and systems of multi-tenant access for a medical record system (e.g., facilitating multiple users editing the medical records) and/or synchronizing records relating to the visit across multiple types of records and/or multiple systems. Before turning to the figures, which illustrate certain examples in detail, it should be understood that the present disclosure is not limited to the details or methodology set forth in the description or illustrated in the figures. The various concepts introduced above and discussed in greater detail below can be implemented in any of numerous ways. It should also be understood that the terminology used herein is for the purpose of description only and should not be regarded as limiting.

During a visit to a healthcare facility or another healthcare environment, a patient may encounter multiple medical professionals (e.g., healthcare receptionists, nurses, medical assistants, physicians, or other clinicians, etc.). For example, the patient may go through a check-in process with a healthcare receptionist, a rooming process with a nurse, and an examination process with a physician, among other processes. The medical professionals may record a variety of information throughout the visit. For example, the healthcare receptionist may collect identity information (e.g., the patient's name, phone number, date of birth, address, insurance information, etc.) that is required before the patient is treated. The nurse may need to record medical information during the rooming process. For example, the nurse may measure and record the patient's vitals (e.g., weight, height, heart rate, blood pressure, etc.) or record/update the patient's medical history. The physician may then need to record medical information that may relate to diagnoses, treatments, care plans, test results, or any other information from the examination process of the visit.

It can be a challenging task to organize all of the notes from the various medical professionals into a consolidated patient record. In some examples, healthcare environments may implement a medical charting system that stores all of the information associated with a patient into a shared electronic medical record, such as check-in information, rooming process information, and examination information, among other information. Some such charting systems may be organized such that all of the patient's information is accessible within, and edited within, one large record. However, such charting systems may be difficult to navigate, as the large amounts of information all in one record can overwhelm the medical professionals, especially if many areas of the record need to be accessed simultaneously. Using this type of charting system may also slow down the patient visit. For example, the information entered by the various medical professionals may not be synchronized to all of the devices running the system in sufficient time. This may cause a delay if some of the notes taken by one medical professional are needed for the next medical professional to proceed with the visit process. Additionally, information entered into the patient's record may be incorrect or lost. If all of the medical professionals are operating on the same system, the same part of the patient's record may be unknowingly changed by multiple different professionals at once, which may result in some of the notes being saved incorrectly.

To overcome these and other challenges, the present disclosure relates to improved medical charting systems and methods. The systems and methods, according to various example implementations, may allow multiple users (e.g., multiple medical professionals associated with a patient's visit with a healthcare provider) to efficiently access notes entered into the patient's record during the visit. The systems and methods may allow the multiple users to edit various parts of the record during the same timeframe. For example, the system may detect that a first user is editing a first section of the record. While the first user is editing the first section, the first section may be locked such that the other users operating on the medical charting system are restricted from editing the first section. Upon completion of editing the first section, the first section may then be unlocked such that all of the multiple users are permitted to edit the first section.

In some implementations, the systems and methods may allow multiple sections to be locked by multiple users during the same timeframe. For example, while the first section is locked by the first user, a second section may be locked by a second user, a third section may be locked by a third user, etc. In some implementations, the edits made to the locked sections may be viewable by the multiple users. For example, the systems and methods may provide a user interface to display any of the sections of the record, including any sections that are locked. While a user is viewing a locked section, the user interface may be updated to display the edits that are made to the locked section.

The systems and methods, according to various implementations, may synchronize edits made by a first user to be viewed by the other users. For example, the first user may edit information (e.g., capture notes) on the record. Responsive to receiving a trigger event, such as an approval of the information by the first user, the information may be stored and pushed to the user interfaces of the other users. The information may be pushed to the user interfaces such that the other users can view the information as it is entered. In some examples, some of the information captured in a first section of the record may be pushed to a second section, such as a condensed health record. The first section and the second section may be provided within the same display screen. Thus, the information captured in the first section may be synchronized to the second section on the same display.

The systems and methods described herein, according to various example implementations, provide technical solutions to these and other challenges. For example, by employing the locking algorithm, examples of the disclosure can reduce the likelihood of medical information being overridden or otherwise entered incorrectly. By preventing such overridden or incorrect information from being entered into a patient visit record, examples of the present disclosure may reduce or eliminate risks associated with incorrect or incomplete information being accessed by the same user or a different user, such as an important note from the nurse not being seen by a doctor. Additionally, the systems and methods, according to various example implementations, can perform a synchronization process, which may allow the users to receive patient information more efficiently, thereby increasing the efficiency of the patient visit.

The present disclosure also provides example implementations that provide enhanced user interfaces, which deliver a particular technical solution that eases navigation of medical records. For example, a navigation portion of the user interface may be provided to allow a user to selectively view a desired portion of the medical record. Additionally, the sections of the medical record are displayed with a common architecture such that the process of entering information is done consistently across sections. The user interfaces are also updated based on the locking algorithm to inform the users of which section is being edited and who is editing it. Additionally, the user interfaces are displayed such that users can access multiple sections and selectively move the displays of information. The user interfaces are updated based on the synchronization of the notes to ensure that the users have access to the most recent patient notes. Accordingly, various pieces of information can be easily referenced while a user is capturing notes.

Various implementations reduce processing power by performing various processing operations simultaneously, rather than performing a plurality of processing operations individually and consuming unnecessary processing power. For instance, the locking algorithm reduces the likelihood of multiple users accidentally editing the same section of information. Thus, the locking process described herein reduces the computational complexity and processing power typically required to screen through the notes to properly validate the notes that should be stored, such as by processing the notes to determine whether there are any conflicts between the notes, where certain notes should be stored in relation to one another in the event of multiple simultaneous edits without locking, etc. Additionally, the synchronization process allows notes captured in one section to populate one or more sections that require similar notes. Thus, the synchronization process described herein reduces the computational complexity and processing power typically required to receive and process the same or similar notes in each individual section.

Various examples additionally or alternatively improve the efficiency of a patient visit, thereby improving the patient's experience during the visit. For instance, the locking process can reduce the likelihood of notes being overridden, thereby reducing the need for parts of the patient visit to be repeated. Additionally, synchronization of the notes across devices allows the different parts of the visit to be performed efficiently. For example, any notes taken during the rooming process may be quickly synchronized to the devices of the medical professionals performing the examination process, such that the examination process does not have to be delayed. Additionally, some examples increase the visit efficiency by generating assistance to help the users conduct the visit more efficiently. For example, recommended health screenings can be generated based on the patient's information, prompts may be generated to help the users walk through a conversation with the patient, scripts may be provided to auto-populate a portion of the notes, billing codes may be generated to assist the users with creating the patient's bill, etc.

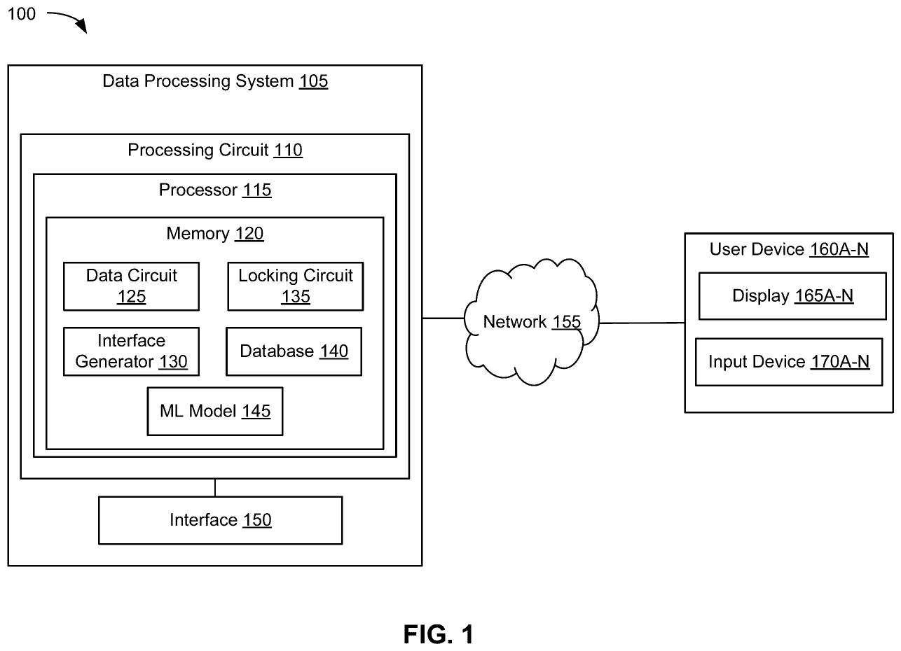

FIG. 1 depicts an example system 100 to capture records for a visit of a patient with a healthcare provider from a plurality of users during the same timeframe. For example, the system 100 can include at least one data processing system 105, a network 155, and one or more user devices 160A-N.

The data processing system 105 can include one or more processing circuits 110. The processing circuit 110 can include one or more processors 115 coupled with memory 120. The processing circuit 110 can include any combination of hardware or software for processing instructions, such as instructions for providing functionalities of the data processing system 105 or data, such as the data of the processor 115, memory 120, data circuit 125, interface generator 130, locking circuit 135, database 140, ML model 145, interface 150, network 155, user device 160A-N, display 165A-N, or input device 170A-N. For example, the processing circuit 110 can receive input data or instructions from a user device 160A. The processor 115 can be implemented as a general-purpose processor, an application specific integrated circuit (ASIC), one or more field programmable gate arrays (FPGAs), an artificial intelligence (AI) processor (such as a neural processing unite (NPU) and tensor processing unite (TPU), a super scalar processor, a group of processing components, or other suitable electronic processing components.

The memory 120 (e.g., memory, memory unit, storage device, etc.) may include one or more devices (e.g., RAM, ROM, flash memory, hard disk storage, etc.) for storing data and/or computer code for completing or facilitating the various processes, layers, and modules described in the present application. The memory 120 can be or include volatile memory or non-volatile memory. The memory 120 can include database components, object code components, script components, or any other type of information structure for supporting the various activities and information structures described in the present application. According to an exemplary example, the memory 120 is communicably coupled to the processor 115 via the processing circuit 110 and includes computer code for executing (e.g., by processing circuit 404 and/or processor 406) one or more processes described herein.

In some examples, the database 140 may include at least one of a computing device, a remote server, a server bank, a remote device, and/or among other possible computer hardware and/or computer software. For example, the database 140 may include a server bank and the server bank can store, keep, maintain, and/or otherwise hold the various types of information described herein. In some examples, the database 140 may house and/or otherwise implement at least one of the various systems, devices, and/or components described herein. In some examples, the database 140 may include, store, maintain, and/or otherwise host the data processing system 105. For example, the data processing system 105 may be distributed across one or more servers (e.g., the database 140). In some implementations, the data processing system 105 and/or various other components of the system 100 may be implemented using cloud computing services/platforms.

The processing circuit 110 can be communicably connected to one or more components of the data processing system 105. For example, the processing circuit 110 can be communicably connected to the interface 150. The interface 150 can include at least one of network communication devices, network interfaces, and/or other possible communication interfaces. The interface 150 can include wired or wireless communications interfaces (e.g., jacks, antennas, transmitters, receivers, transceivers, wire terminals, etc.) for conducting data communications with various systems, devices, and/or components described herein. The interface 150 can be direct (e.g., local wired or wireless communications) and/or via a communications network (e.g., the network 155). For example, the interface 150 can include an Ethernet card and port for sending and receiving data via an Ethernet-based communications link or network. The interface 150 can also include a Wi-Fi transceiver for communicating via a wireless communications network (e.g., the network 155). The interface 150 can include a power line communications interface. The interface 150 can include an Ethernet interface, a USB interface, a serial communications interface, and/or a parallel communications interface.

Memory 120 is shown as including several components, including the data circuit 125, the interface generator 130, the locking circuit 135, the database 140, and the ML model 145. In some examples, these components may be implemented as instructions stored within the memory 120 or one or more other computer-readable storage media that are executable by the processor(s) 115 to perform various functions described herein. In some implementations, the components may additionally or alternatively be implemented in part or in whole using hardware. All such alternatives are contemplated within the scope of the present disclosure.

In some implementations, the data circuit 125 may receive records to be stored and processed. For example, the data circuit 125 may receive edits (e.g., edits 210) from one or more user device(s) and process the edits 210 to be readable by the other components of the data processing system 105. The data circuit 125 may be configured to detect errors in the received edits 210 and validate at least a portion of the edits 210. The data circuit 125 may store the received edits 210 and other records to the database 140.

In some implementations, the interface generator 130 may provide a user interface. For example, the interface generator 130 can provide the user interface to a display 165A on a user device 160A. The interface generator 130 may update the user interface to display one or more records that are recorded by the plurality of users of the user devices 160A-N. The interface generator 130 may also generate user interfaces that are configured to receive the records from the user devices 160A-N.

In some implementations, the locking circuit 135 may detect that a user (e.g., a receptionist, a nurse, a clinician, a radiographer, etc.) is editing a section of the patient record. For example, the locking circuit 135 may receive an indication from one of the input devices 170A-N that a user is editing a section. In response to detecting the user is editing the section, the locking circuit 135 may lock the section to locking by other users. The locking circuit 135 may unlock the section after the edits to the section have been completed.

The memory 120 may store, hold, keep, hold, or otherwise maintain at least one machine learning (ML) model 145, in some implementations. The ML model 145 may be trained using one or more various ML and/or Artificial Intelligence (AI) techniques. For example, the ML model 145 may be trained using supervised and/or unsupervised learning. As another example, the ML model 145 may be trained using deep learning techniques. One or more components of the data processing system 105 may access and/or utilize the ML model 145. For example, the processors 115 may utilize the ML model 145. The ML model 145 may be trained to generate one or more relevant user interfaces, or portions of one or more interfaces, depending on the context of the patient visit. For example, the ML model 145 may provide a list of relevant screenings or questionnaires relevant to the patient on the display 165A-N.

In some examples, the network 155 may include at least one of a local area network (LAN), a wide area network (WAN), a telephone network (such as the Public Switched Telephone Network (PSTN)), a Controller Area Network (CAN), a wireless link, an intranet, the Internet, a cellular network, and/or combinations thereof. In some examples, the various systems, components, and/or other devices included in the system 100 may communicate with one another via the network 155. In some examples, the network 155 may include a network 155a and a network 155b. The network 155a and the network 155b may refer to separate portions within the network 155. For example, the network 155a may receive records (e.g., edits 210) from one of the user devices 160A-N and facilitate the transfer of the edits to the data processing system 105 for processing, validation, and storing. The network 155a may be or include a microservice that is designated for transmitting the edits 210. The network 155b may be configured to transmit records to the plurality of user devices 160A-N. In some examples, the network 155b may include a pipeline to distribute information to the various user devices 160A-N. In some examples, the network 155b may include a database 157. The database 157 may be separate from the database 140. The database 157 may be a cloud-based database, such as a data lake. The database 157 may be used for large-scale storage to store large volumes of patient data accumulated over time. The database 157 is shown in a process 200, as described further herein.

In some examples, the user devices 160A-N may perform various actions and/or access various types of information. The information may be provided over the network 155. In some examples, the user devices 160A-N may perform similar operations to that of the system 100. For example, the user devices 160A-N may perform similar operations to that of the data processing system 105. The user devices 160A-N may include one or more applications to receive information, display information, and/or receive user interactions with content displayed by the user devices 160A-N. The user devices 160A-N may receive information from a user on input devices 165A-N. For example, the input devices 165A-N may be or include a mobile computing device, a desktop computer, a smartphone, a tablet, a smart watch, and/or any other device that can facilitate receiving and otherwise interacting with content. Information may be displayed on the user devices 160A-N on displays 170A-N. For example, the displays 170A-N may be or include a screen, a monitor, a visual display device, a touchscreen display, a television, a video display, a liquid crystal display (LCD), a light emitting diode (LED) display, and/or any other device that can facilitate providing, displaying, and otherwise interacting with content. The user devices 160A-N may provide one or more inputs to the data processing system 105.

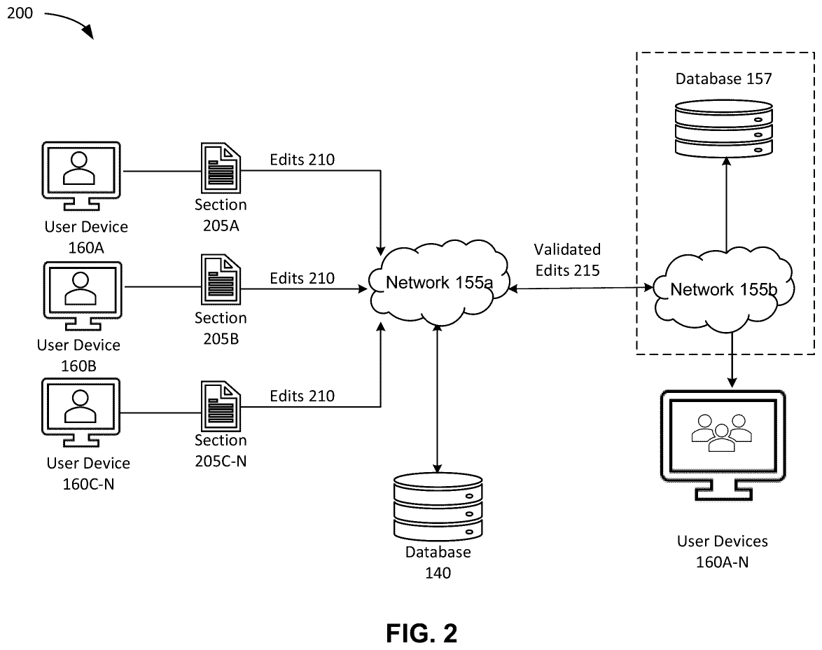

FIG. 2 depicts a flow diagram of a process 200, according to some examples. In some examples, the process 200 may represent and/or illustrate one or more steps, actions, processes, and/or transmissions to receive, process, store, display and/or otherwise capture records for a visit of a patient with a healthcare provider from a plurality of users during the same timeframe. The process 200 and/or one or more portions thereof may be executed by and/or carried out by the system 100 and/or one or more systems, devices, components, and/or elements thereof. For example, the processing circuits 110 may execute the process 200. It should be understood that the process 200, as described further herein, may be performed for any of the sections 205A-N of information on the patient record. For example, section 205A, as described in process 200, may represent any of the sections 205A-N (i.e., section 205A as described in the process 200 is not explicitly limited to being or including the visit purpose section 205A).

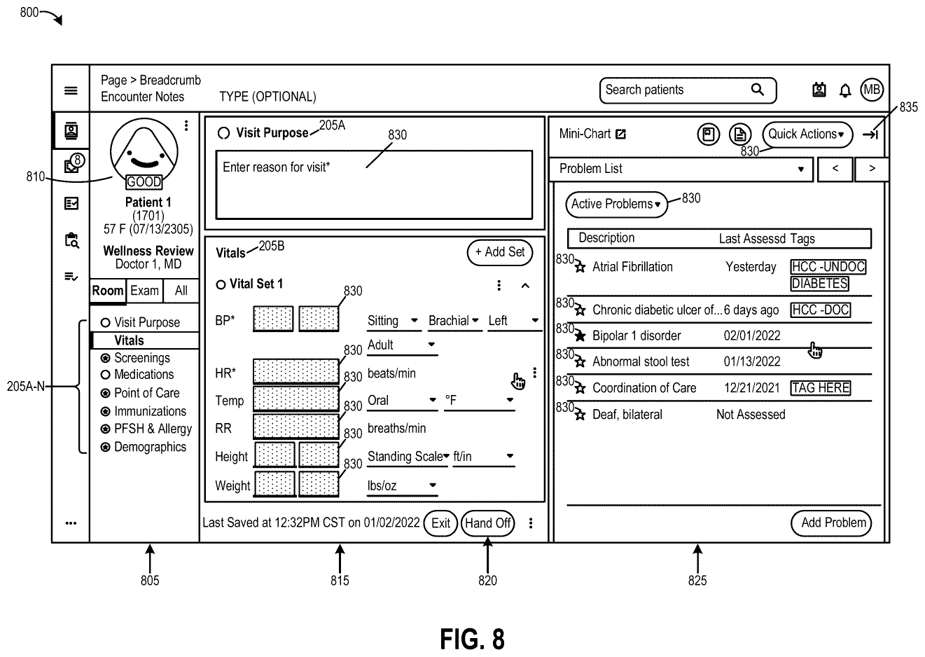

Referring now to FIGS. 1 and 2, in some examples, the interface generator 130 may provide user interfaces to a plurality of users (e.g., users operating the user devices 160A-N). For example, the interface generator 130 may provide a user interface displaying a plurality of sections 205A-N that capture information regarding at least one of the patient or the visit. The interface generator 130 may provide interface 800, shown in FIG. 8, to display a general overview of the sections 205A-N on the user devices 160A-N and/or provide selectable icons or other elements to allow the user to view the sections 205A-N.

In some examples, the locking circuit 135 may manage locking of sections while a user is editing the sections, such that one or more other users may be prevented from making edits while the user is making edits. In some implementations, the locking circuit 135 may detect that a first user is editing a first section. For example, the locking circuit 135 may detect that edits 210 to section 205A are being made on the user device 160A. Responsive to detecting that the edits 210 are being made, the locking circuit 135 may lock the section 205A. When locked, section 205A may only be editable by the user of the user device 160A and edits may not be permitted from the other user devices 160A-N. In some implementations, the locking circuit 135 may lock editing by some other users while the section is locked but may permit edits from some other users (e.g., users having certain permissions, roles, etc.).

Edits 210 to other sections 205A-N may be received within the same timeframe as the edits to section 205A. For example, the locking circuit 135 may detect that edits 210 to section 205B are being made on the user device 160B. Responsive to detecting that edits 210 are being made, the locking circuit 135 may lock the section 205B. When locked, the section 205B may only be editable by the user of the user device 160B and edits may not be permitted from one or more other user devices 160A-N. In some implementations, the locking circuit 135 may prevent edits from being made by any of the other user devices 160A-N when the section 205B is locked. As another example, the locking circuit 135 may detect that edits 210 to section 205C are being made on the user device 160C. Responsive to detecting that the edits 210 are being made, the locking circuit 135 may lock the section 205C. When locked, the section 205C may only be editable by the user of the user device 160C and edits may not be permitted from other user devices 160A-N. Any of the sections 205A-N may be locked and receive edits from their respective user device 160A-N during one timeframe. For example, during a patient visit for a first patient, only user device 160A may have one of the sections 205A locked for editing. As another example, during a patient visit for a second patient, five of the user devices A-N may have five different sections 205A-N locked for editing. As used herein, edits can be within the same timeframe, simultaneously, concurrently, contemporaneously, etc., if at least a portion of the edits occur at the same time as the other edits, even if the entire timeframes during which the edits are made in the different sections does not overlap. It should be appreciated that, where the present disclosure describes that a section is locked and edits cannot be made by other user devices, in various implementations, the edits may be prohibited or prevented from all other user devices or just some of the user devices, and all such implementations are contemplated within the scope of the present disclosure.

In some implementations, the data circuit 125 may collect the edits 210 to the sections 205A-N. For example, the data circuit 125 may receive the edits 210 to section 205A from the user device 160A through the network 155, such as the network 155A. In some examples, the edits 210 may be received by the interface 150. The data circuit 125 may process the edits 210 such that they may be stored in the database 140. The database 140 may store all the edits 210 to the section 205A. For example, if the edits 210 require a validation to be pushed to the user interface, the database 140 may store both the edits 210 that have and have not received the validation. In this way, any of the edits 210 that have not been completed (e.g., the user is interrupted while taking notes) will be stored and accessible for future use. The data circuit 125 may collect multiple notes at least partially concurrently. For example, the edits 210 to at least one of section 205B, 205C, or any other section locked and edited, may be collected, and stored in the database 140 in the same timeframe as section 205A.

In some implementations, the data circuit 125 may validate the edits 210. The data circuit 125 may receive a validation from one of the user devices 160A-N. For example, the data circuit 125 may receive the validation to the edits 210 of the section 205A from the user of the user device 160A. The validation may also be generated by the data circuit 125. For example, the data circuit 125 may be configured to validate the edits 210 based on the data received in the edits 210. For example, if the edits 210 in a field for a blood pressure reading only include a systolic value and does not include a diastolic value, the data circuit 125 may determine that the edits 210 are invalid. In some examples, the data circuit 125 may access previously stored information, such as a predetermined value range, from a data structure (e.g., the database 140 or the database 157), to validate the edits 210. For example, if the edits 210 in a field for a medication dosage exceed the possible dosage range, the data circuit 125 may determine that the edits 210 are invalid. In some examples, the ML model 145 may be trained to validate the edits 210. For example, if the edits 210 in a field for a medication dose include a 1000 mg dose for a medication that is only given in a 100 mg dose, the ML model 145 may determine that the edits 210 are invalid.

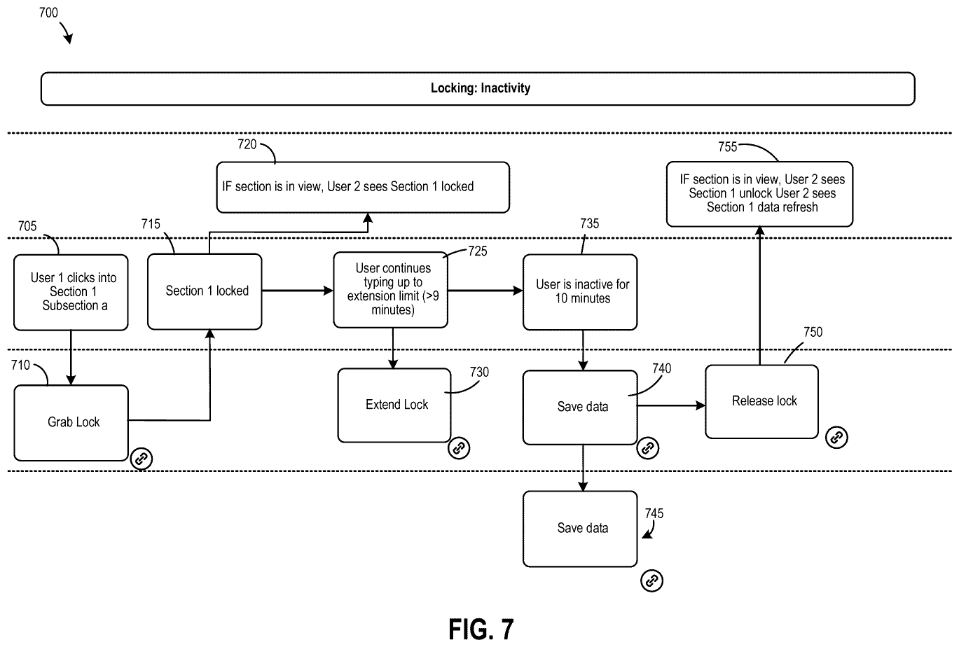

In some implementations, the data circuit 125 finalizes the edits 210 responsive to detecting a trigger event. Finalizing the edits 210 may refer to the validation process performed by the processing circuit 110. Additionally, or alternatively, finalizing the edits 210 may refer to confirming, by the processing circuit 110, that the edits 210 are complete (e.g., the user has finished editing). In this example, the edits 210 are only stored in response to detecting the trigger event. The trigger event may refer to the validation from one of the user devices 160A-N. For example, the user interface of the user editing the section 205A may display a selectable element (e.g., a save button, a confirmation button, etc.) that is configured to receive the validation from the input device 170A. The trigger event may refer to an editing timeout event, which is illustrated in FIG. 7 in greater detail. For example, if the user editing the section 205A has not edited the section 205A in over 10 minutes, the locking circuit 135 may unlock the section 205A. The locking circuit 135, or another component in the data processing system 105 may transmit the trigger event responsive to unlocking the section. Additionally, or alternatively, the trigger event may be transmitted in response to any unlocking process, and is not limited to the editing timeout event.

The records, including the validated edits 215, may be transmitted to the network 155, such as the network 155B, to update the user interface. For example, the interface generator 130 may update a portion of the user interface that displays the section 205A to display the records associated with the section 205A, including the validated edits 215 from the user device 160A. The updated user interface displaying the validated edits 215 may be transmitted to all of the user devices 160A-N. For example, if a user of the user device 160D is currently viewing the section 205A, the user interface will be updated to display the validated edits 215 once validated. In this way, the user viewing section the 205A does not need to refresh the user interface to receive the validated edits 215. As another example, if a user of the user device 160E is currently viewing another one of the sections 205A-N, the validated edits 215 will still be pushed to the user device 160E. In this way, the section 205A including the validated edits 215 will be available for display. In some examples, the network 155B may also store the validated edits 215 in the database 157.

In some examples, the notes captured in one of the sections 205A-N may be used to additionally update one or more of the sections 205A-N. For example, the validated edits 215 may have been added to the section 205A. The validated edits 215 may include a list of the patient's allergies. The validated edits 215 may then be added, by the processing circuit 110, to a second section 205B. For example, the section 205B may include medication information, where a portion of the required medication information includes the patient's allergies. As such, the allergy list stored in section 205A may also be stored in section 205B.

The user interface may display the records of the sections 205A-N within a threshold time relative to when the edits 210 are received. For example, the edits 210 to the section 205A may be received by the interface 150. The interface generator 130 may then update the user interface of the user devices 160A-N to display the records of the first section 205A, including the validated edits 215, within 30 seconds from being received by the interface 150. The threshold time may be shorter or longer, such as 10 seconds, 5 seconds, 45 seconds, 1 minute, or any other predetermined threshold time defined in the system 100. In some examples, the threshold time refers to the time between the validated edits 215 being validated and the user devices 160A-N receiving the validated edits 215.

In other examples, the sections 205A-N may need to be updated on the user devices 160A-N to display the records. For example, the validated edits 215 may be stored to at least one of the databases 140 or 157 within the threshold time described above. However, the interface generator 130 may require a prompt to generate a new user interface displaying the records of the section 205A, including the validated edits 215. For example, the prompt may be or include an input, such as a selection of a refresh icon, from the user device(s) 160A-N that are viewing the section 205A. Using the prompt to update the user interface may mitigate any confusion associated with the records being automatically pushed to the section 205A.

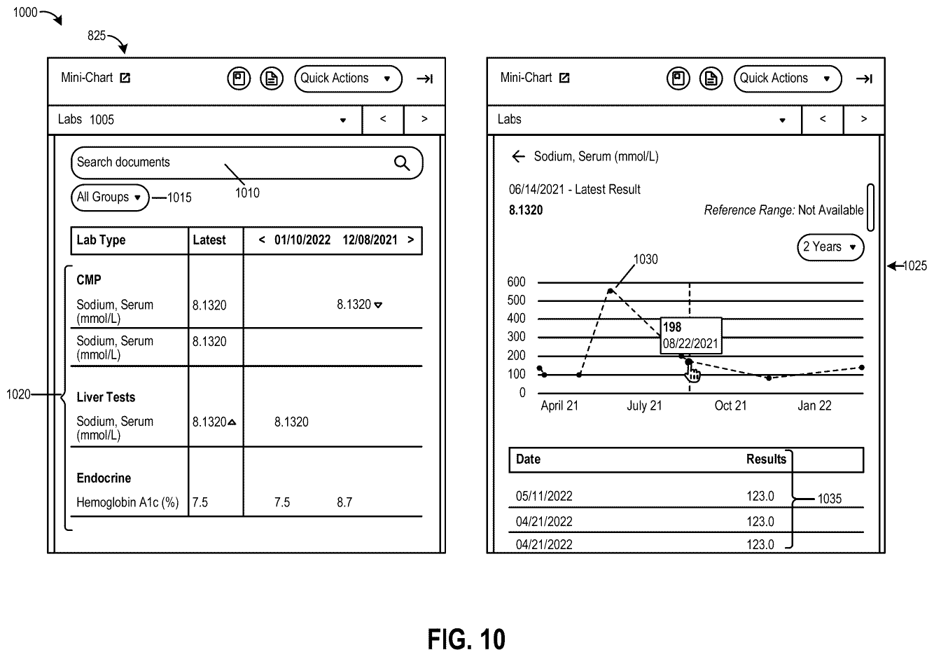

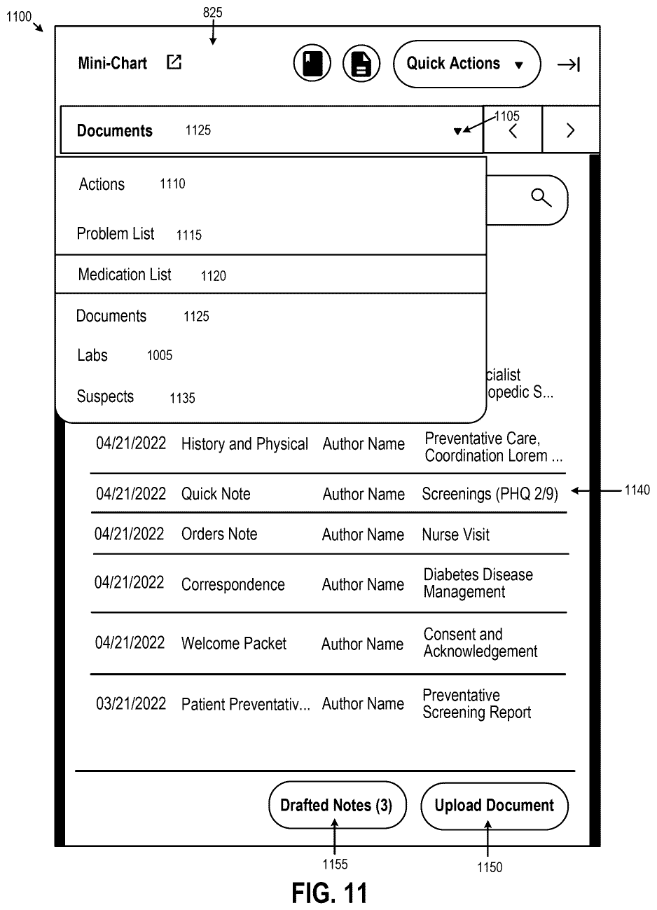

In some examples, the user interface may include a plurality of portions provided on the same display 165A-N. For example, the user interface may display a first portion that includes the section 205A that is being edited by the user of the user device 160A. The second portion may include a condensed health record 825 of the patient. The condensed health record 825 may refer to a data overview relating to the patient that summarizes all of the patient data across sections 205A-N. For example, the condensed health record 825 may display an active problems list, lab information, a medications list, and other relevant data relating to the patient or the visit. FIGS. 10-11, among others, as described further herein, illustrate the condensed health record 825 in further detail.

In some examples, the sections 205A-N may be synchronized with the condensed health record 825. For example, in addition to updating the first portion on the user interface (e.g., the portion including the section 205A), the processing circuits 110 may transmit at least a portion of the records, including the edits to the section 205A, to at least a portion of the condensed health record 825 of the patient. For example, the records in section 205A may include 10 vitals values collected by a nurse and notes relating to each vital value. Once validated, 5 of the vitals that are required for the condensed health record 825 may be transmitted to the designated portions of the condensed health record 825. The condensed health record 825 may then be updated with the relevant records and displayed on the user devices 160A-N that are currently viewing the condensed health record 825. The condensed health record 825 may be automatically updated. For example, the validated edits from section 205A may be populated into the condensed health record without an additional input from the user, such that updates to the section 205A and the condensed health record 825 are synchronized. In some examples, the condensed health record 825 may be updated without locking the condensed health record 825. For example, the records from sections 205A, 205B, 205C, or any other locked sections 205A-N being edited in the timeframe may be used to populate the condensed health record 825 in that timeframe. The synchronized updates to the condensed health record 825 may be pushed to any of the user devices 160A-N that are viewing the condensed health record 825. The records, including the validated edits 215 to the sections 205A-N, may be synchronized within a threshold time. The threshold time may refer to a time after the edit 210 is received, a time after the validated edit 215 is validated, among other threshold times. In one example, the pushing of the update is done within a threshold time after the edit 210 is received. This threshold time may be 1 minute, 30 seconds, 15 seconds, or any other threshold time.

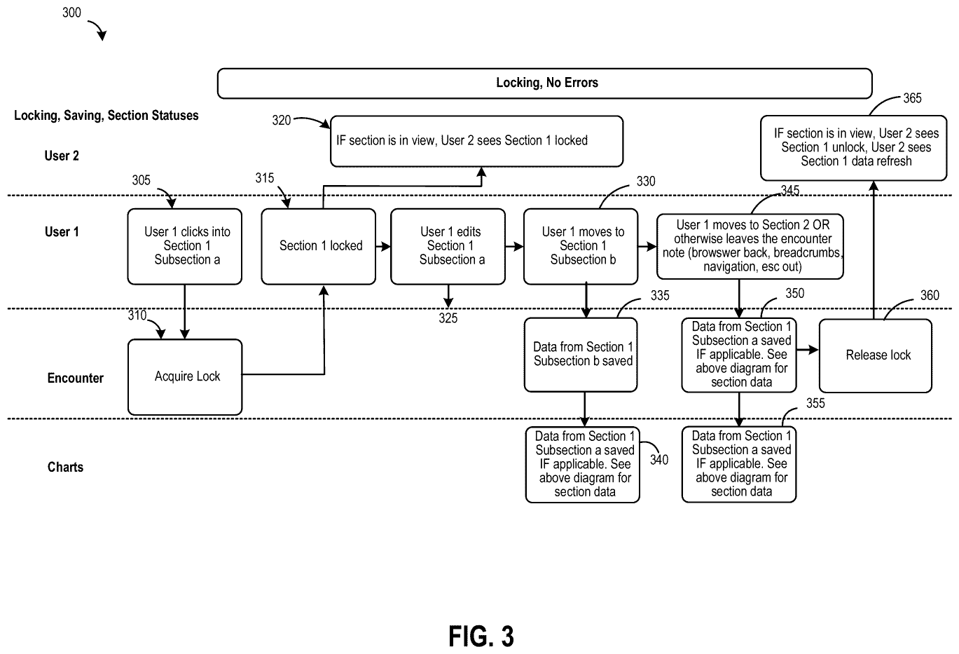

In some implementations, the locking circuit 135 may unlock the locked sections 205A-N after the edits have been made. For example, the locking circuit 135 may detect that the user of the user device 160A has finished editing the section 205A. Responsive to detecting that the user has finished editing the section 205A, the locking circuit 135 may unlock the section 205A. Once unlocked, all of the users of the user devices 160A-N are permitted to edit the section 205A. Similarly, the locking circuit 135 may unlock section 205B, 205C, or any other sections 205A-N that were locked in response to the respective users completing the edits. When another user makes edits to the section 205A, or another section 205A-N that has been unlocked, the section 205A will be relocked by the locking circuit 135. FIGS. 3-7, as described further herein, illustrate the locking and unlocking process in greater detail.

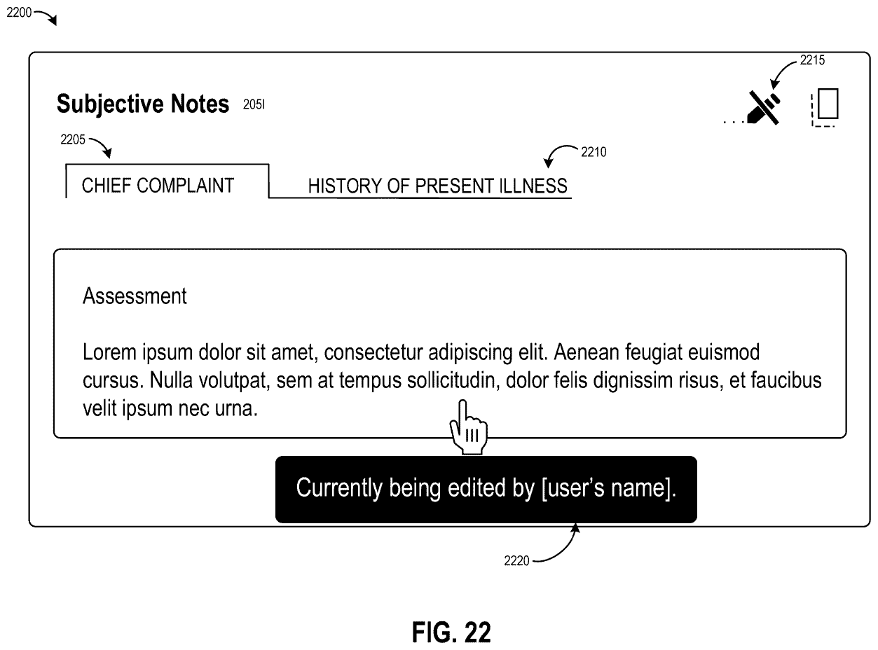

In some implementations, the interface generator 130 may provide an indication of a locking status of the sections 205A-N. The indication may include a lock icon, a colored icon, or any other indication used to indicate that the section, such as section 205A, is locked. The indication may include identity information. For example, when section 205A is locked by the user of user device 160A, the interface may provide identity information to the user interface for the other users of the user devices 160A-N. The identity information may be a name, an identification number/code/name (e.g., employee identifier), a profile picture, an abbreviation, or any other information that indicates an identity of the user of the user device 160A. The identity information may be used to display, to the other users, who is editing the section 205A. When the section 205A is unlocked by the locking circuit 135, the interface generator 130 may display an indication that the section 205A has been unlocked. For example, an unlocked icon, a colored icon, or any other indication to indicate that section 205A is unlocked, may be displayed. Additionally, or alternatively, the locked icons and/or the identity icons may simply be removed from the user interface to indicate that the section is unlocked. The user interface 2200 of FIG. 22, as described further herein, illustrates the indications of the locking status in greater detail.

As described further herein, the user interface may include a plurality of sections 205A-N that are each configured to capture and display different information. The database 140, or another location in the data processing system 105, may store the captured records from each section 205A-N in a designated location (e.g., the database 140 may include a section 205A of the database 140). A portion of sections 205A-N are associated with a rooming process of the patient visit. The rooming process refers to the process taken by clinical staff (e.g., a nurse, medical assistant, receptionist, or other clinical staff) to prepare for the examination by the doctor. For example, the sections 205A-H may be associated with the rooming process. A portion of the sections 205A-N are associated with an examination process of the patient visit. The examination process refers to the process performed by the physician or other medical professional to assess the patient's health and diagnose/treat conditions.

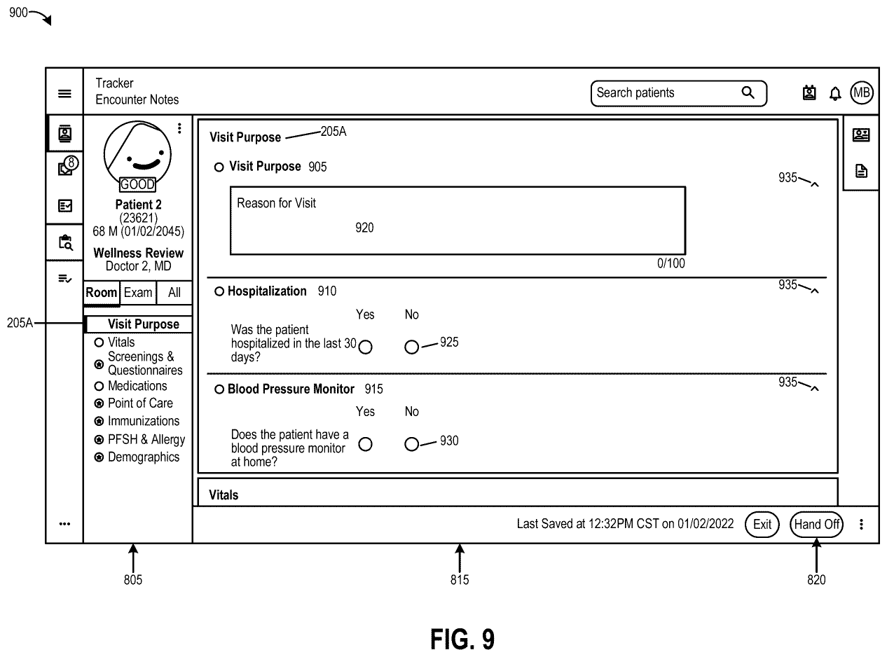

The sections 205A-N may include a visit purpose section 205A. The processing circuits 110 may receive, store, and display visit purpose information for the patient via the visit purpose section 205A. For example, the visit purpose section 205A may be configured to receive, store, and display notes relating to the reason for the visit, hospitalization information related to the patient, blood pressure information related to the patient, among other general visit purpose information regarding the patient. The visit purpose section 205A may be edited by a user (e.g., a nurse or a medical assistant) during the rooming process of the visit. The visit purpose section 205A and further details are shown in the user interface 900 of FIG. 9, as described further herein.

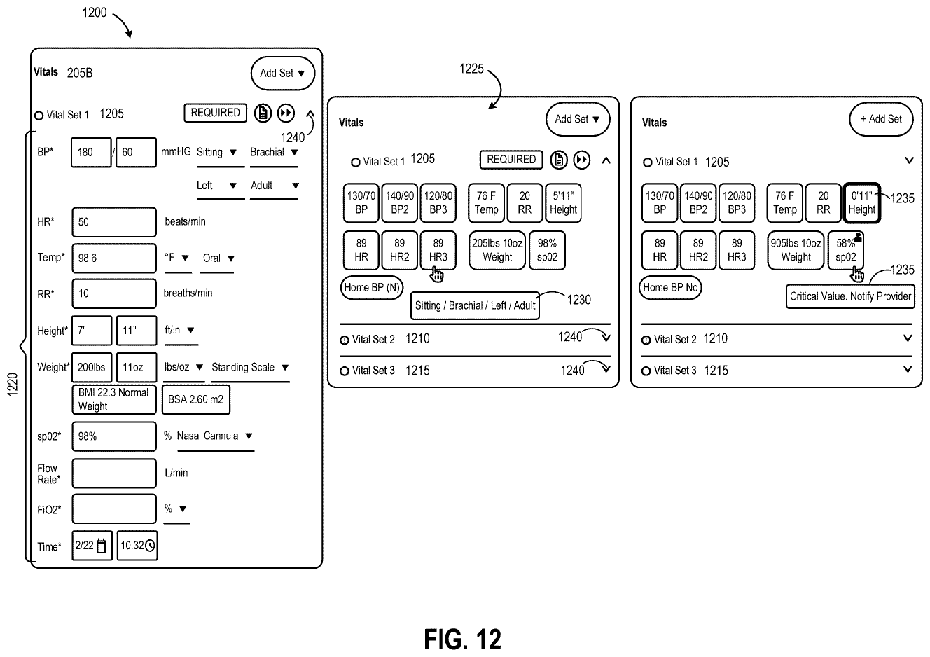

The sections 205A-N may include a vitals section 205B. The processing circuits 110 may receive, store, and display vitals information for the patient via the vitals section 205B. For example, the vitals section 205B may be configured to display a blood pressure, a heart rate, a temperature, a respiratory rate, a height, a weight, a body mass index (BMI), a body surface area (BSA), an oxygen saturation, a flow rate, a fraction of inspired oxygen, and/or any other vitals associated with the patient. The vitals section 205B may be edited by a clinical staff user during the rooming process of the visit. The vitals section 205B and further details are shown in the user interface 1200 of FIG. 12, as described further herein.

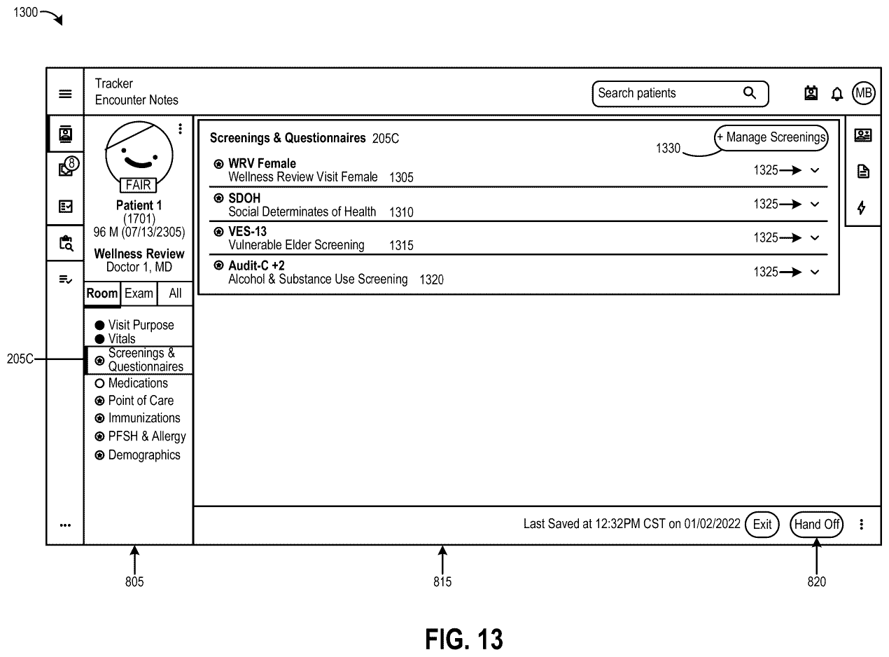

The sections 205A-N may include a screening/questionnaire section 205C. The screening section 205C may receive, store, and display information about screenings relating to the patient. For example, the screening section 205C may include a list of required screenings for the patient, recommended screenings for the patient, and results of previously performed screenings. In some examples, the ML model 145 may generate a list of relevant screenings for the patient. For example, the ML model 145 may retrieve the stored data of the patient, such as records from previous visits or records currently being captured. Using the patient data, the ML model 145 may identify the most relevant screenings that should be done for the patient. Additionally, or alternatively, the screenings may be selected on the user device(s) 160A-N. The screening section 205C and further details are shown in the user interfaces 1300 of FIGS. 13 and 1400 of FIG. 1400, as described further herein.

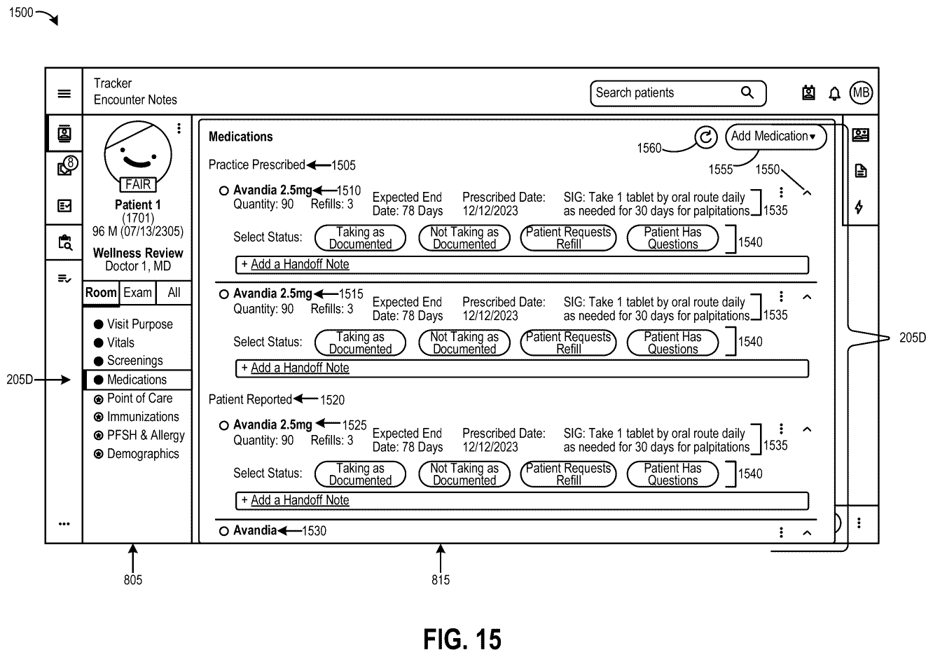

The sections 205A-N may include a medications section 205D. The medications section 205D may store medication information, and the stored medication information may be used to generate a user interface to display the medications section 205D. The medications section 205D may display and collect information, such as detailed information of a prescribed medication (e.g., strength, supply, refills, route, patient instructions, frequency of dose, prescription date, pharmacy notes, and other medication notes). The medications section 205D may store and display a status of a medication, such as current reported concerns, whether a refill is needed, whether the medication was discussed in the rooming process, among other status information. In some examples, the data circuit 125, or another component of the data processing system 105, may recognize an error in a medication record and prevent the record from being updated. For example, the data circuit 125 may detect that a medication added in the medications section 205D is listed on the patient's allergy list. As such, the data circuit 125 will not allow that medication to be updated to the patient record. The medications section 205D and further details are shown in the user interface 1500 of FIG. 15, as described further herein.

The sections 205A-N may include a point of care section 205E. The point of care section 205E may be used to receive and update updated records from the rooming process, such that the updated records are stored and displayed. In this way, a user can enter partial records, with a note providing a reason for incomplete records, that will be displayed and updated once the records are completed. In some examples, the interface generator 130 will update the user interface with a time that the records were updated, such that the users are informed that the records have been changed.

The sections 205A-N may include an immunizations section 205F. The immunizations section 205F may be used to receive and display immunization information associated with the patient. For example, the immunization sections 205F may receive an order for a vaccination and link the order to an administered vaccine. The immunization section 205F may include documentation of a vaccination status (e.g., administered, not given, past dose, refused, etc.). The immunization section 205F may include documentation of a date/time associated with an administration of a vaccine. A questionnaire relating to one of the immunizations may be displayed on the user interface for the user to reference. For example, the questionnaire may relate to information that is needed before the patient receives the immunization. The records in the immunizations section 205F may be transferred. For example, the immunization records may be transmitted, through the network 155, to an immunization tracking server (e.g., a state immunization tracking website).

The sections 205A-N may include an allergy section 205G. The allergy section 205G may be used to receive and display allergy information associated with the patient. The allergy section 205G may include any allergies that the patient has reported, a severity relating to the allergies, any potential allergies, and/or any other information stored, or records captured relating to the patient's allergies. Additionally, or alternatively, the allergy section 205G may include past family social history (PFSH) information. For example, the PFSH information may include general social history, substance abuse history, miscellaneous history, medical history, surgical history, family disease history, or any other PFSH. In some examples, scripts may be provided to assist the users with collecting allergy/PFSH information. For example, the ML model 145 may generate a script, including points of conversation or questions, that will promote the efficiency of capturing the records. The ML model 145 may generate the script based on previously stored patient information from the patient or other patients (e.g., the ML model 145 may generate the script based on a successful script used for a similar patient).

The sections 205A-N may include a demographics section 205H. The demographics section 205H may be used to receive and display demographics information associated with the patient. For example, the demographics section 205H may receive and store information about the patient's education, sexual orientation, race, ethnicity, or preferred language, as well as basic information, such as the patient's name, age, date of birth, and gender. A component of the data processing system 105, such as the ML model 145, may generate a script to promote the efficiency of capturing the patient's demographics information. For example, if the patient reports using cigarettes, the ML model 145 may generate a script to assist with obtaining more detailed information on the patient's cigarette use, based on various factors such as the patient's age and medical history.

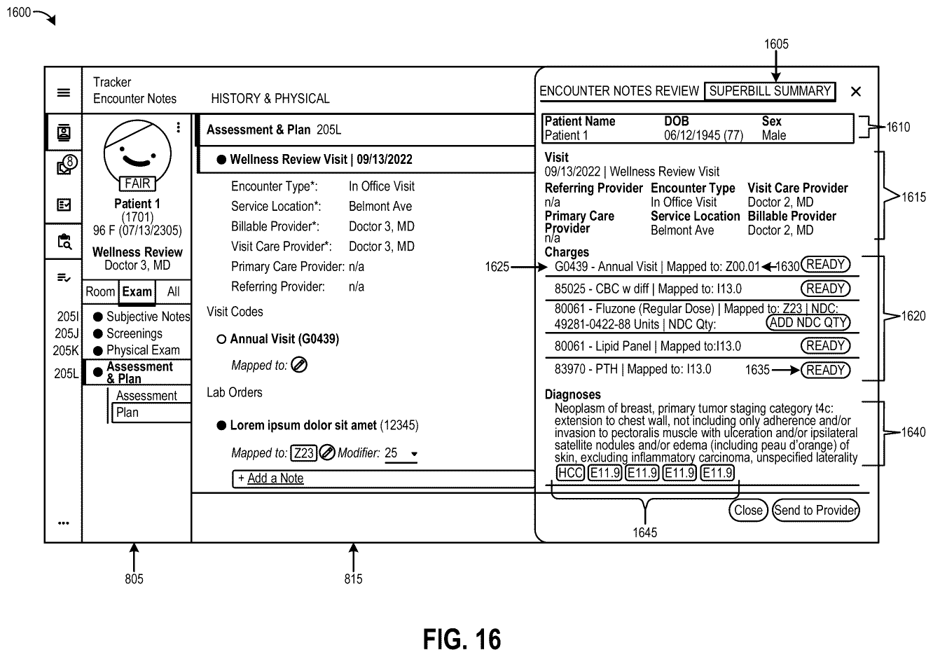

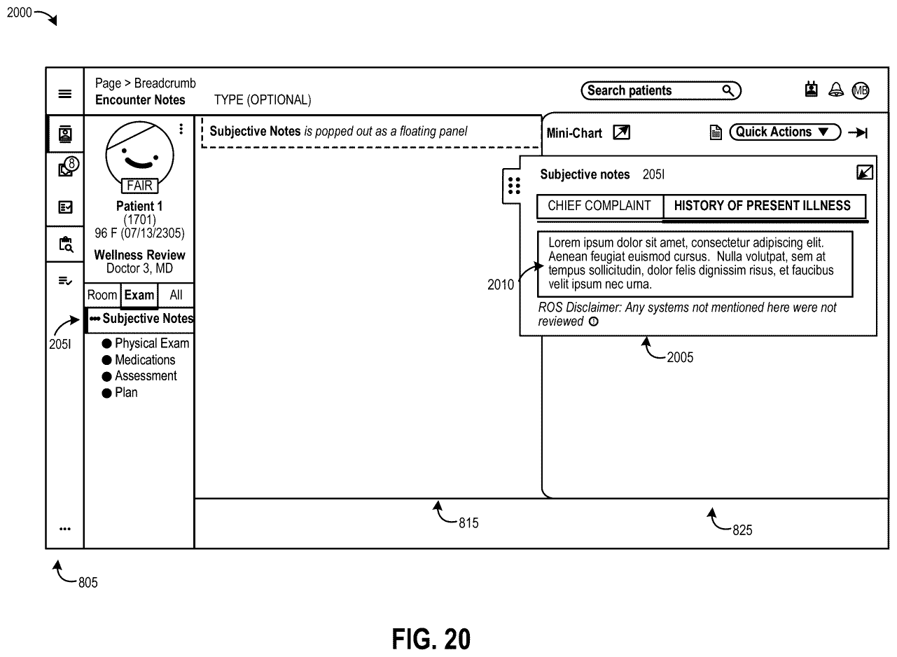

A portion of the sections 205A-N are associated with an examination process of the patient visit. In one exemplary example, sections 205I-L may be used to receive, store, display, and otherwise interact with information relevant to the examination process of the visit. The examination sections 205I-L may include a subjective notes section 205I, which is illustrated with greater detail in FIGS. 20-22. The subjective notes section 205I may be displayed on the user interface as a floating panel to allow the user to input informal records while viewing another one of the sections 205A-N. The examination sections 205I-L may include a second screenings section 205J. The second screenings section 205J may be used to receive, store, display, and otherwise interact with screenings for the patient that are not conducted in the rooming process. The examination sections 205I-L may include a physical exam section 205K. The physical exam section 205K may be displayed, such that the user is able to add physical exam notes as free text. In some examples, prompts may be provided to assist the user with entering the notes. The examination sections 205I-L may include an assessment and plan section 205L, which is illustrated with greater detail in FIGS. 16-19.

In some examples, the sections 205A-N that are displayed on the user interface may be determined by a role of the user. For example, the interface generator 130 may be configured, such that it can only generate displays of information for the user if the user's role is associated with the information. For example, the user of user device 160A may have a role of a nurse (or another medical professional involved in the rooming process). In this example, the nurse may only have access to viewing the rooming process sections 205A-H. As another example, the user of the user device 160B may have a role of a doctor (or another medical professional involved in the examination process). In this example, the doctor may be given access to viewing all of the sections 205A-N. In some examples, the roles of the user may further restrict the viewing permissions. For example, the user of a user device 160C may have a role of a receptionist, who may be involved in the rooming process. Although associated with the rooming process, the receptionist may only be given access to the visit purpose section 205A, or another limited portion of the rooming process sections 205A-H. The role of each user may be determined based on a credential received by the user. For example, the credential may be an employee ID number, a security ID code, a username and password combination, or a QR code, among other credentials. In some implementations, the processing circuit 110 may then determine a comparison between the credential received and information previously disposed in a data structure (e.g., database 140 and/or database 157) relating to medical professional role tags. Using the comparison, a role can be assigned to the user. Additionally, each of the sections 205A-N of information may be associated with one or more role tags. Accordingly, the processing circuit 110, or another component of the data processing system 105, may identify at least a portion of the sections 205A-N that are associated with the user's role tag, and allow the identified portion of the sections 205A-N to be accessed.

In some examples, the locking circuit 135 may permit editing to one of the sections 205A-N by multiple users. For example, section 205A may be locked by a user of the user device 160A. Once locked, the processing circuit 110 may receive or determine the role of the user. For example, the processing circuit 110 may determine that the user's role is a physician. The locking circuit 135 may then allow edits to the section 205A by users having a certain role. For example, a user device 160B for a medical scribe may be given permission to edit the section 205A, while the section 205A is locked by the physician. As such, the physician and the medical scribe are both permitted to edit the locked section 205A. As another example, a section 205C may be locked by a nurse. In response, the locking circuit 135 may allow editing to the section 205C to one or more users having a role with a higher priority level, such as users with roles of a physician or a physician assistant. In this example, a priority level of a role may be determined by information previously stored in a data structure (e.g., database 140 or database 157) or determined by the ML model 145. In some examples, the permissions to access one of the locked sections 205A-N is determined by the processing circuit 110. For example, the ML model 145 may be trained to identify patterns of the user's roles and accordingly give editing permissions to the appropriate users. In other examples, the permissions to access one of the locked sections 205A-N may be received by one of the input devices 170A-N. For example, an input device 170A may receive an indication from a physician operating the user device 160A to allow all users with a “nurse” role to edit their locked section 205A.

FIGS. 3-7 depict processes 300-700, according to some examples. In some examples, the processes 300-700 may represent and/or illustrate one or more steps, actions, processes, and/or transmissions of the locking process. The processes 300-700 and/or one or more portions thereof may be executed by and/or carried out by the system 100 and/or one or more systems, devices, components, and/or elements thereof. For example, the processing circuits 110 may execute the processes 300-700.

FIG. 3 depicts a flow diagram of a process 300 to illustrate one or steps of the locking process with no errors, according to some examples. For example, the process 300 illustrates the process of a first user editing one of the sections 205A-N, such as the section 205A.

The first user may click into a subsection of the section 205A, referred to as subsection 206A. For example, the locking circuit 135 may detect an input, from the input device 170A, to the subsection 206A (action 305). Responsive to detecting the input, the locking circuit 135 may lock the section 205A (action 310). The user interface may display the indication that section 205A is locked. For example, the interface generator 130 may display the indication to the first user that they have section 205A locked for editing (action 315). If a second user has the section 205A in view on their display, the interface generator 130 may display the indication to the second user that the section 205A is locked for editing (action 320).

Once the section 205A is locked, edits 210 may be received. For example, the edits 210 to the subsection 206A may be received from the input device 170A by the network 155 (action 325). The user may then proceed to another subsection. For example, the locking circuit 135 may detect an input, from the input device 170A, to a second subsection, referred to as subsection 207A (action 330). Responsive to proceeding to subsection 207A, the edits 210 to subsection 206A may be saved and validated. For example, the data circuit 125 may store the edits 210 to the database 140 and validate the edits 210, according to the process 200 (action 335). The validated edits 215 may then be used to update a portion of the user interface, according to process 200 (action 340).

The first user may exit the section 205A. For example, the first user may enter another one of the sections 205A-N, escape out of the section 205A, or otherwise exit the section 205A (action 345). Responsive to exiting the section 205A, the edits 210 to the subsection 207A may be validated and stored, according to the process 200 (action 350). The validated edits 215 may then be used to update a portion of the user interface, according to process 200 (action 355). Responsive to storing the edits 210, the locking circuit 135 may unlock the section 205A (action 360). Once the section 205A is unlocked, the interface generator 130 may update the user interface to indicate that section 205A has been unlocked (action 365). In some examples, action 365 may include updating the user interface to display the records, including the validated edits 215, added to section 205A.

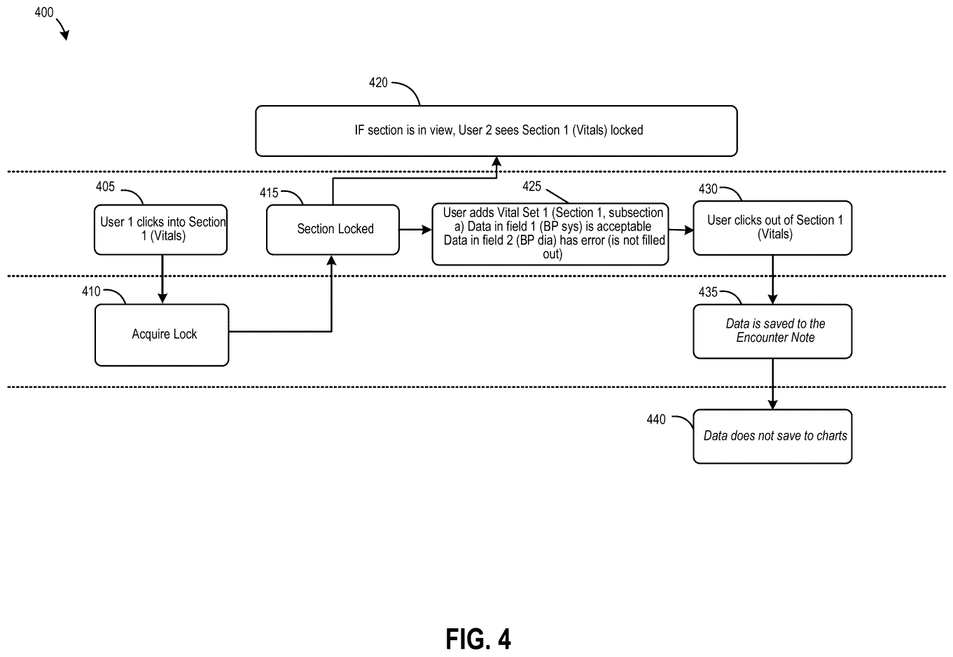

FIG. 4 depicts a flow diagram of a process 400 to illustrate one or more steps of the locking process when invalid data is received, according to some examples. The process 400 receives notes that are at least partially invalid. In response to receiving invalid notes, the notes may be stored and/or saved to the section 205A. In some examples, the condensed health record 825 will not be updated at all in response to receiving notes that are at least partially invalid. In other examples, the condensed health record 825 may be updated with a valid portion of the at least partially invalid notes.

Similar to actions 305-320 of the process 300, the process 400 may include actions 405-420 to lock a section, such as section 205A, and indicate that section 205A is locked for editing. The data circuit 125 may then receive invalid data from the input device 170A (action 425). For example, edits 210 including vitals for a patient may be received by the input device 170A. The vitals may include an acceptable first field (e.g., a systolic blood pressure value) and an invalid second field (e.g., a diastolic blood pressure value is missing). The first user may then exit the section 205A (action 430, performed similarly to action 345). The edits 210 may then be stored (action 435). For example, the data circuit 125 may store the edits 210 in at least one of the databases 140 or 157. In some examples, the edits 210 may be saved within the section 205A, but not pushed to the condensed health record 825. The edits 210 are not saved to the condensed health record 825, as they are not valid (action 440). In some examples, the condensed health record 825 may be partially updated with the edits 210 at action 440. For example, if two fields of edits 210 are received and the data circuit 125 determines that one of the two fields are valid, those validated edits 215 may be used to update a portion of the condensed health record 825.

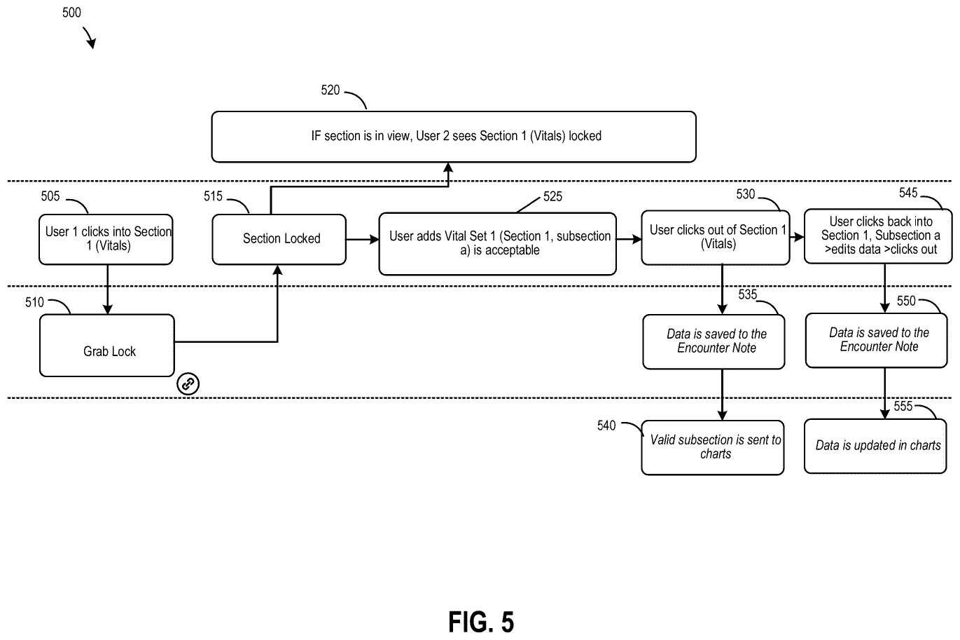

FIG. 5 depicts a flow diagram of a process 500 to illustrate one or more steps of the locking process when data is updated after being received, according to some examples. The process 500 includes locking a section to editing, receiving valid notes, populating the user interface with the valid notes, and updating the user interface based on updates made to the section.

Similar to actions 305-325 of the process 300, the process 500 includes actions 505-525 to lock a section (e.g., section 205A), indicate that the section 205A is locked for editing, and receive edits 210 to the section 205A. The user may then exit the section 205A (action 530, performed similarly to action 345). Responsive to exiting the section 205A, the data circuit 125 may validate the edits 210 and store the validated edits 215 (action 535). The interface generator 130 may then generate an updated interface to display the condensed health record 825, including a portion of the validated edits 215 (action 540). The user may then re-enter the section 205A and generate new edits 210 to change the validated edits 215 (action 545). Once stored and validated, the new validated edits 215 may be updated to the section 205A (action 550). Additionally, the condensed health record 825 may be updated according to the new validated edits (action 555).

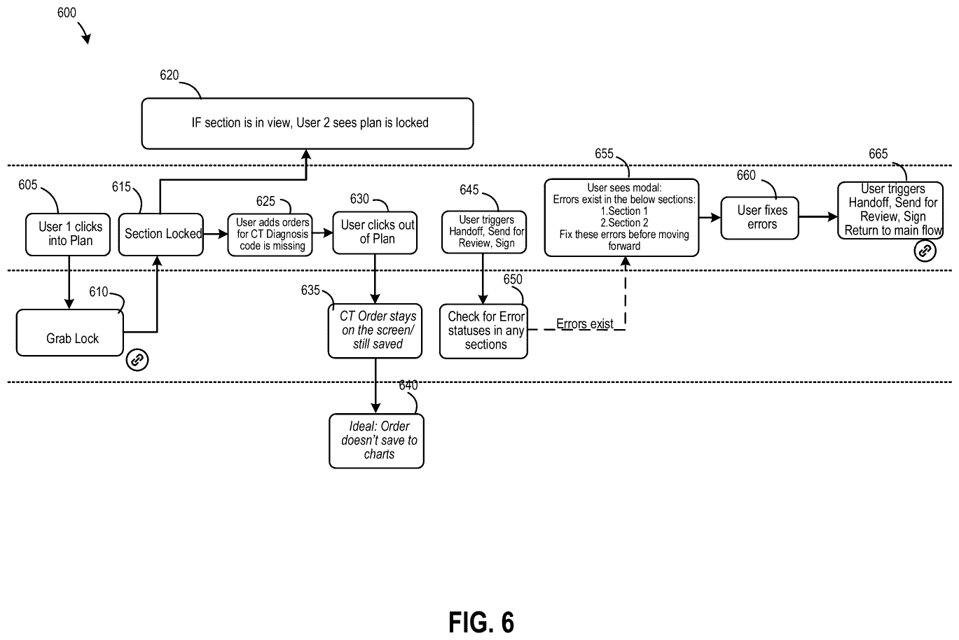

FIG. 6 depicts a flow diagram of a process 600 to illustrate one or more steps of the locking process with a handoff, according to some examples. The process 600 includes locking a section (e.g., the plan) to editing, adding a lab order, and triggering a handoff to transmit the records.

Similar to actions 305-325 of the process 300, the process 600 includes actions 605-625 to lock a section (e.g., the plan section), indicate that the plan section is locked for editing, and receive the edits 210 to the plan (e.g., an order for a CT scan). The user may then exit the plan (action 630, performed similar to action 345). Regardless of whether the edits 210 were incorrect (e.g., a diagnoses code was missing in the CT scan order), the edits 210 may be saved. Additionally, the edits 210 may remain on the display 165A of the user device 160A (action 635). The edits 210 will not be updated to the user interface (action 640). In some examples, action 640 is only performed when the edits 210 have errors. In other examples, the action 640 is performed regardless of the validity of the edits 210. The user may then trigger a handoff of the plan. For example, the processing circuit 110 may receive an indication that a handoff element on the user interface has been selected (action 645). Responsive to triggering the handoff, the processing circuit 110 may process the record, including the edits 210, to check for errors (action 650). In the event that there are still errors in the record, the user interface for the user will provide an indication that errors are present (action 655). Once the errors are fixed (action 660), the handoff can be re-triggered. Upon successful triggering of the handoff, the plan, including the edits 210, are transmitted to another user. The process 600 may repeat and/or omit actions. For example, if the edits 210 received in action 625 do not have errors, the process 600 may omit actions 640, 655, and/or 660.

FIG. 7 depicts a flow diagram of a process 700 to illustrate one or more steps of the locking process when a user is inactive, according to some examples. The process 700 may include locking a section (e.g., section 205A) to editing, and unlocking the section 205A when the user is inactive on the user device 160A.

Similar to actions 305-320 of the process 300, the process 700 includes actions 705-720 to lock a section (e.g., the section 205A) and indicate that the section 205A is locked for editing. In some examples, the locking circuit 135 is configured to extend the lock. For example, edits 210 or other activity may be received by the user device 160A for an amount of time that is past an extension limit (action 725). The extension limit may be greater than five minutes, greater than 10 minutes, or any other desired extension limit. Responsive to exceeding the extension limit, the locking circuit 135 may extend the lock on the section 205A such that the lock is not disabled prematurely (action 730). Additionally, or alternatively, the user device 160A may be inactive for a predetermined time (action 735). For example, the data processing system 105 may not receive any activity from the user device 160A for 10 minutes, five minutes, or any other predetermined time. In that event, the edits 210 that were obtained over the entirety of the locking duration are saved to the section 205A (action 740) and saved to the condensed health record (action 745). Once the edits 210 are saved, the locking circuit 135 may release the lock (action 750). The interface generator 130 may update the user interface for the user devices 160A-N to indicate that the section has been unlocked (action 755).

FIGS. 8-24 depict various user interfaces generated by the interface generator 130. In some examples, the system 100 employs a unified architecture for the user interfaces. For example, the layouts (e.g., the header, the portion to display information, the shape of selection icons, etc.) are, at least partially, consistent between the sections 205A-N. In this way, capturing records, viewing records, or otherwise interacting with the patient record is done similarly between the sections 205A-N, which may reduce the time that a user takes to navigate the system 100. In some examples, the user interfaces include scroll-lock headers. For example, the user interface 1500 may have a header to indicate the medication section 205D. The header may remain viewable on the display as a user scrolls through the section, reducing the risk that the user enters information into an incorrect section. Additionally, or alternatively, each of the sections 205A-N may include multiple scroll-lock headers throughout the sections. For example, the vitals section 205B may include three scroll-lock headers corresponding to each of the required vital sets for the patient.

FIG. 8 depicts a user interface 800 with a general patient record overview, according to some examples. In some examples, the user interface 800 may be generated, displayed, and/or represented responsive to a record of a selected patient being selected. In some examples, the user interface 800 may include three portions on the same display. The first portion may be or include a navigation portion 805. The navigation portion 805 may include a general patient overview 810. For example, the general patient overview 810 may include at least one of a name, an identification number, an age, a birthday, an associated physician, or a profile picture for the patient. The navigation portion 805 may include one or more selectable icons associated with each of the plurality of sections 205A-N. For example, the user interface 800 illustrates a portion of the plurality of sections 205A-N that may be associated with the rooming process of the patient visit. The visit purpose section 205A, the vitals section 205B, the screenings portion 205C, the medications section 205D, the point of care section 205E, the immunizations section 205F, the allergy section 205G, and the demographics section 205H are shown as an example of rooming process sections. The selectable elements associated with the sections 205A-N may be used to display one of the desired sections 205A-N to a user.

The second portion of the user interface 800 may include a notes portion 815. In some examples, the notes portion 815 may display the desired sections 205A-N, as selected in the navigation portion 805. For example, user interface 800 displays the visit purpose section 205A and the vitals portion 205B in the notes portion 815. In some examples, the notes portion 815 may display several of the sections 205A-N (e.g., the rooming sections 205A-H, the examination sections 205I-L, or all sections 205A-N), which may be listed in the order that they are to be completed during the visit. In this example, the user interface 800 adjusts the visible sections 205A-N based on a scrolling position. The notes portion 815 may include one or more selectable elements 830 to receive edits 210. The one or more selectable elements may be buttons, text fields to receive text, selectable drop-down menus, or any other element configured to receive edits 210. The notes portion 815 may include a hand off element 820. For example, the hand off element 820 may send an indication to the data processing system 105 to transmit the notes taken in the displayed section(s) 205A-N to another user.

The third portion of the user interface 800 may include a condensed health record 825. The condensed health record 825 may include selectable elements 830, such as selectable icons, drop-down menus, text fields to receive text, or any other elements configured to navigate through portions of the condensed health record 825 and/or receive notes relating to the condensed health record 825. In some examples, the condensed health record 825 is collapsible. The condensed health record 825 may include a minimizing element 835. When selected, the minimizing element 835 may at least partially hide the condensed health record 825 and expand the notes portion 815 for viewing and/or editing.

FIG. 9 depicts a user interface 900 to illustrate the visit purpose section 205A, according to some examples. In some examples, the selectable element for the visit purpose section 205A may be selected from the navigation portion 805. Once selected, the notes portion 815 may display at least a portion of the contents of the visit purpose section 205A. For example, the user interface 900 displays a general visit purpose portion 905, a hospitalization portion 910, and a blood pressure monitor portion 915. The user interface 900 may include one or more selectable elements 920-930 to receive visit purpose information. For example, a text box 920 may be included to receive notes in the general-purpose portion 905, a series of buttons 925 may be included to answer questions within the hospitalization portion 910, and a series of buttons 930 may be included to answer questions within blood pressure monitor portion 915. The portions 905-915 may be expandable and collapsable for viewing. For example, each portion 905-915 may include a collapse element 935 that is configured to selectively collapse or expand its respective portion 905-915. The user interface 900 may include the handoff element 820.

FIG. 10 depicts a user interface 1000 to illustrate a portion of the condensed health record 825, including a labs portion 1005, according to some examples. The labs portion 1005 may include lab information, lab results, lab dates, lab result trends, and/or any other lab information pertaining to the patient. The user interface 1000 may include one or more selectable elements to navigate the labs portion 1005. For example, the selectable element 1010 may be a search bar configured to receive text to search through the lab information. As another example, the selectable element 1015 may be a drop-down element 1015 to provide selectable filters to sort the lab information. The user interface 1000 may include a list of labs 1020. For example, the list of labs 1020 may include at least a portion of the labs given to and/or ordered for the patient. The list of labs 1020 may include a summary of the results for each lab, such as lab name, latest results, and dates performed, among other information. The user interface 1000 may be configured to display a selected lab in greater detail, shown as lab information 1025. The lab information 1025 may provide greater detail for the lab, such as a chart 1030 to show a trend for results or a list of dates and corresponding results 1035, among other details for the lab tests.

FIG. 11 depicts a user interface 1100 to illustrate a portion of the condensed health record 825, including a documents portion 1125, according to some examples. The documents portion 1125 may include a list of documents 1140. The list of documents 1140 may display at least a portion of the stored documents associated with the patient and may be configured to allow the user to select a document to view. The documents portion 1125 may include an upload selection 1150 to allow a user to upload a new document. The documents portion 1125 may also include a drafted notes selection 1155 to allow the user to view drafted documents that have not been uploaded, or drafted notes to be referenced when writing a formal document.

Illustrated on the user interface 1100, the condensed health record 825 may include a selectable element, such as a drop-down menu element 1105, configured to navigate between the portions of the condensed health record. For example, the condensed health record 825 may include at least one of the labs portion 1005, an actions portion 1110, a problems portion 1115, a medication portion 1120, the documents portion 1125, or a suspects portion 1135, among others. In the user interface 1100, the documents portion 1125 is selected from the drop-down menu element 1105 and displayed.

FIG. 12 depicts a user interface 1200 to illustrate the vitals section 205B, according to some examples. The vitals section 205B may include multiple vital sets, shown as vital set 1205, vital set 1210, and vital set 1215. Each of the vital sets 1205-1215 may include a plurality of selectable elements 1220 to receive patient vitals. For example, the selectable elements 1220 may be text boxes to receive text entries, drop-down menus to select units or other parameters associated with the vitals, and/or any other elements to receive any information relating to the vitals. The user interface 1200 illustrates selectable elements 1220 to receive information for each one of the following vitals: blood pressure, heart rate, temperature, respiration rate, height, weight, BMI, BSA, oxygen saturation, flow rate, fraction of inspired oxygen, and time that the vitals were measured. The user interface 1200 may display a summarized view of the vitals after they are recorded. For example, vitals summary 1225 summarizes the vitals in the vital set 1205. The vitals summary 1225 may be configured, such that additional information 1230 is made viewable, such as when a cursor is hovered over a vital. The vitals summary 1225 may include a warning indication 1235 to emphasize critical vital values. For example, the oxygen saturation value in the vitals summary 1225 may be colored to indicate a critical value. Additional information on the critical value may be made viewable, such as a warning message being displayed when a cursor hovers over the warning indication 1235. The warning indication 1235 may additionally emphasize vital values that may be incorrect or otherwise invalid. For example, the height in the vitals summary 1225 may be colored to indicate a potentially invalid height value. The user interface 1200 may include a collapsable icon 1240 to selectively expand and/or collapse portions of the vitals section 205B.

FIG. 13 depicts a user interface 1300 to illustrate the screening section 205C, according to some examples. In some examples, the selectable element for the screening section 205C may be selected from the navigation portion 805. Once selected, at least a portion of the screening section 205C may be displayed in the notes portion 815. For example, the notes portion 815 may include a screenings list including some or all of the screenings 1305-1320 associated with the patient. The screenings list may include the screenings recommended by the ML model 145, screenings recommended by one of the users, screenings required for all patients, screenings required for the specific patient, or any other screenings recommended or required for the patient. For example, the screenings list may include a female wellness review 1305, a social determinates of health questionnaire (SDOH) 1310, a vulnerable elder screening 1315, an alcohol and substance use screening 1320, and/or any other screenings or questionnaires relating to the patient. The user interface 1300 may include a collapsable element 1325 configured to selectively expand or collapse one of the screenings 1305-1325. The user interface 1300 may include a manage screenings element 1330 to add or remove a screening, edit the notes associated with one of the screenings, or otherwise interact with the information within the screening section 205C. The user interface 1300 may include the hand off element 820 to transmit the notes taken in the screening section 205C to another user.

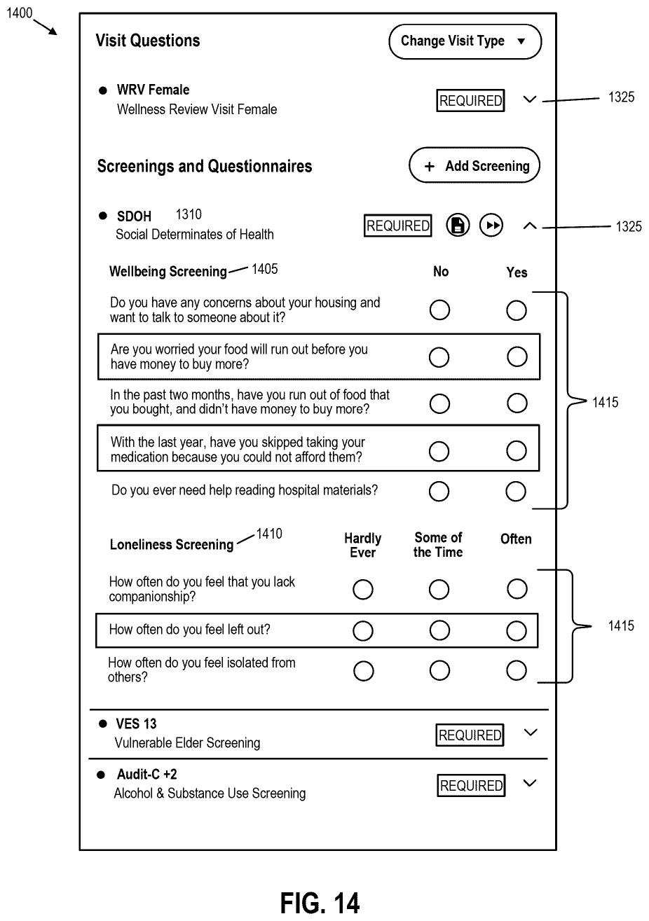

FIG. 14 depicts a user interface 1400 to illustrate a screening in the screening section 205C, according to some examples. The collapsable element 1325 may be selected to expand one of the screenings 1305-1320, such as the SDOH 1310. In the expanded view, the user interface 1400 may illustrate at least a portion of the questions associated with the SDOH 1310, shown as wellbeing screening questions 1405 and loneliness screening questions 1410. Each of the wellbeing screening questions 1405 may have a respective selectable element 1415 configured to receive an answer to the questions 1405. Each of the loneliness screening questions 1410 may have a respective selectable element 1415 configured to receive an answer to the questions 1410.

FIG. 15 depicts a user interface 1500 to illustrate the medications section 205D, according to some examples. In some examples, the selectable element associated with the medications section 205D may be selected from the navigation portion 805. Once selected, at least a portion of the medications section 205D may be displayed in the notes portion 815. For example, a practice prescribed medications list 1505 and a patient reported medications list 1520 may be displayed. The practice prescribed medications list 1505 may list any medications that were prescribed by the physician in the system, shown as medications 1510 and 1515. The patient reported medications list 1520 may list any medications that are reported by the patient, such as medications 1525 and 1530. Each of the medications 1510-1530, along with any other medications listed, may include a medication summary 1535. The medication summary 1535 may include information related to the medication and/or prescription, such as a prescription quantity, an amount of refills, an expected end date, a prescribed date, and administration instructions, among any other relevant medication information. Each of the medications 1510-1530, along with any other medications listed, may include a medication status 1540 to indicate a status of the medication. For example, the medication status 1540 may indicate whether the patient is taking the medication(s) 1510-1530 as documented, whether the patient requests a refill, or whether the patient has questions, among other relevant status information. The user interface 1500 may include a collapsable element 1550 to selectively expand or collapse a portion associated with one of the medications 1510-1530. An add medication element 1555 may be included to allow the user to add a new medication associated with the patient. A refresh button 1560 may be included to allow the user to refresh the user interface 1500 to include any changes in the medications section 205E.

FIG. 16 depicts a user interface 1600 to illustrate the superbill summary 1605, according to some examples. The superbill summary 1605 may include a patient information summary 1610. For example, the patient information summary 1610 may include patient information such as a name, date of birth, sex, and/or any other relevant information associated with the patient. The superbill summary 1605 may include a visit summary 1615. For example, the visit summary 1615 may include information relating to the visit such as a referring provider, an encounter type (e.g., in person visit, telehealth visit, etc.), a visit care provider, a primary care provider, a service location, a billable provider, and/or any other information relevant to the visit. A list of charges 1620 may be included to display the charges associated with the visit. The charges 1620 may include an annual visit charge, a blood test charge, a medication charge, a lab charge, among others. The visit code 1625 mapped to each charge may be displayed next to its respective charge 1620. The billing code 1630 mapped to each charge may be displayed next to its respective charge 1620. The visit codes 1625 and billing codes 1630 may include those generated by the ML model 145 or may be selected by the user. A billing status 1635 may be displayed near its respective charge 1620. For example, the annual visit charge 1620 may have a ready status 1635 to indicate that it is ready to be billed. The superbill summary 1605 may include a diagnoses section 1640 to summarize the diagnoses associated with the patient. The diagnoses codes 1645 may be displayed in the diagnoses section 1640. The diagnoses codes 1645 may be those determined by the ML model 145 and/or selected by the user. The diagnoses codes 1645 may be used in other processes, such as recommending questionnaires, recommending medications, generating prompts for formal records, etc.

The user interface 1600 shows an additional plurality of the sections 205A-N that are associated with the examination process of the patient visit. For example, a subjective notes section 205I, a second screenings section 205J, a physical exam section 205K, and an assessment and plan section 205L section, among other sections 205A-N associated with the examination process may be displayed on the user interface 1600. User interface 1600 shows a display where the assessment and plan section 205L is selected for display. As such, an example of at least a portion of the assessment and plan section 205L is displayed in the notes portion 815.

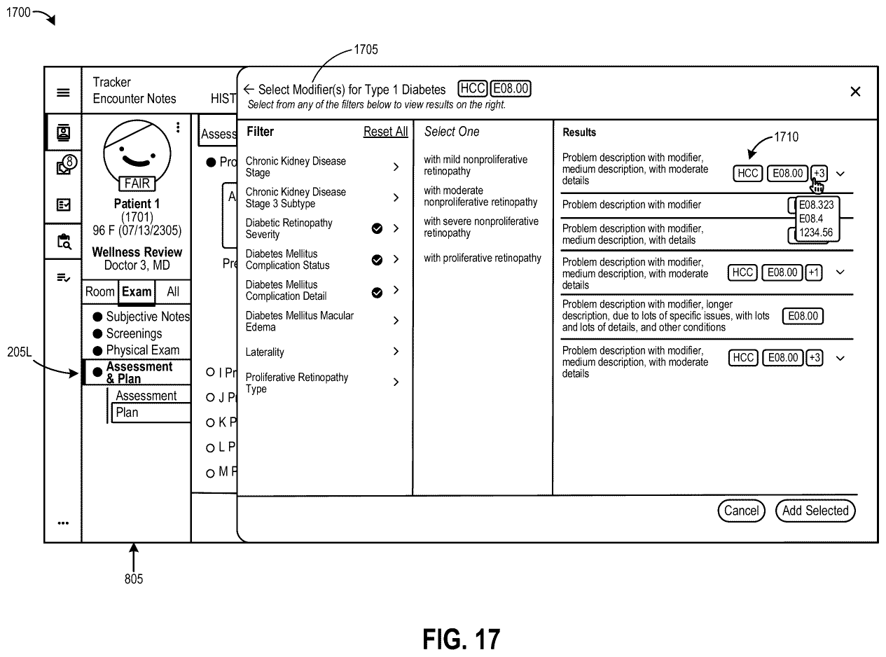

FIG. 17 depicts a user interface 1700 to illustrate a portion of the assessment and plan section 205L, including a diagnoses modifier portion 1705, according to some examples. In some examples, the selectable element associated with the assessment and plan section 205L may be selected from the navigation portion 805. At least a portion of the assessment and plan section 205L includes diagnoses modifiers 1705. For example, if the patient has Type 1 Diabetes, the user interface 1700 may display filters associated with Type 1 Diabetes to classify the patient into a more precise diagnoses category. The user interface 1700 may display codes 1710 that are associated with the precise diagnoses categories. The relevant codes 1710 may be selected. The codes 1710 may be used to create the superbill summary 1605, generate screening or questionnaire recommendations, or in other areas of the patient record.

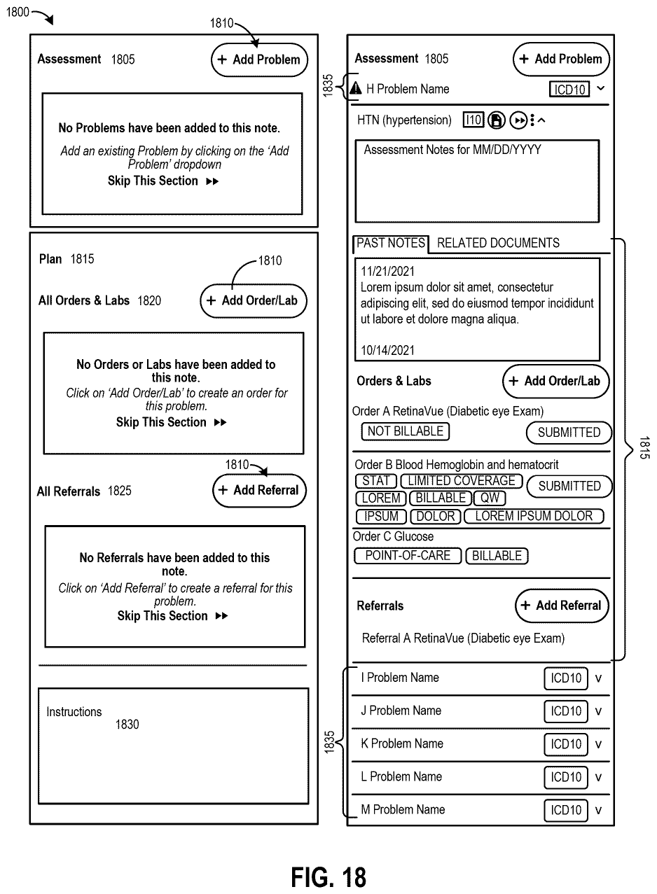

FIG. 18 depicts a user interface 1800 to illustrate a portion of the assessment and plan section 205L, according to some examples. The assessment plan section 205L may include an assessment portion 1805. The assessment portion 1805 may include a selectable element 1810 to allow a user to add a problem for the patient. For example, the selectable element 1810 is configured to receive an input from the user related to a new problem for the patient. The assessment portion 1805 may also display any existing problems associated with the patient. The assessment plan section 205L may include a plan portion 1815. The plan portion 1815 may include an orders and labs portion 1820. The orders and labs portion 1820 may include a selectable element 1810 to receive an input from the user related to any lab orders for the patient. The orders and labs portion 1820 may also display any existing orders associated with the patient. The assessment and plan section 205L may include a referrals portion 1825. Similar to the portions 1805 and 1820, the referrals portion 1825 may receive an input relating to referrals associated with the patient and display existing referrals. For example, the referrals portion 1825 may receive an input relating to referrals for the patient. The assessment and plan section 205L may also include an instructions portion 1830. The instructions portion 1830 may be a text box configured to receive instructions relating to the assessment and plan from the user. The assessment and plan section 205L may include a separate assessment portion 1805 and plan portion 1815 for each of the problems added for the patient, shown as the problems list 1835.

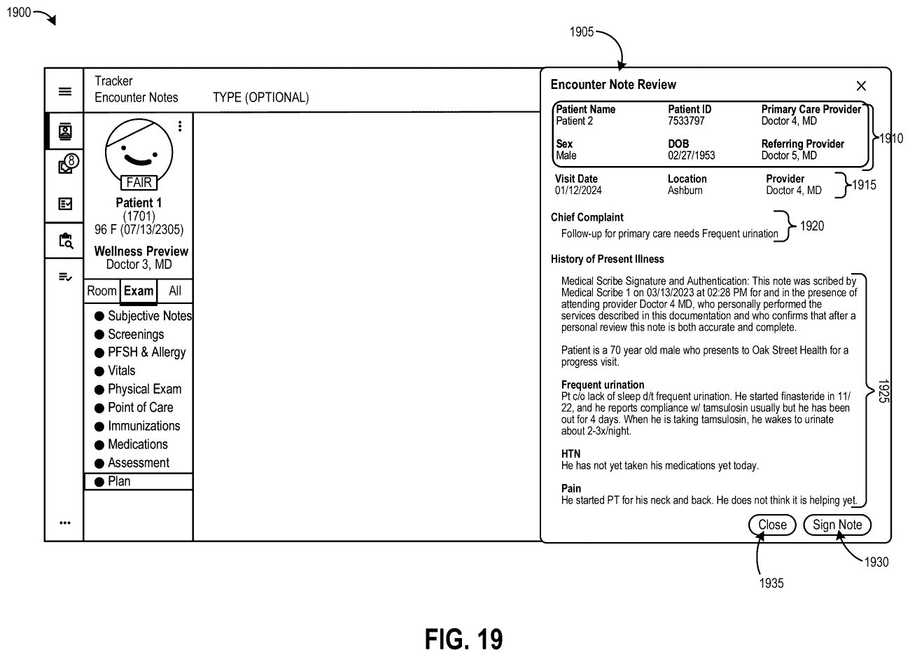

FIG. 19 depicts a user interface 1900 to illustrate a record review 1905, according to some examples. The record review 1905 may include a patient summary 1910. The patient summary 1910 may include patient information such as a name, patient ID, primary care provider, sex, date of birth, or referring provider, among other patient information. The record review 1905 may include a visit summary 1915. The visit summary 1915 may include visit information such as a visit date, a visit location, a provider, among other visit information. The record review 1905 may include a chief complaint summary 1920 to summarize the patient's reason for the visit. The record review may include a history summary 1925. For example, the history summary 1925 may include history of the patient issue, medication history, pain history, among other patient history. A confirmation element 1930 may be included and configured to store and transmit the record when selected. A cancel element 1935 may be included and configured to allow the user to update the records before confirming. The record review 1905 may be displayed to allow the record, including any edits made, to be reviewed before it is saved and transmitted to the user devices 160A-N.

FIG. 20 depicts a user interface 2000 to illustrate the subjective notes section 205I, according to some examples. In some examples, the selectable element associated with the subjective notes section 205I may be selected from the navigation portion 805 of the user interface. Once selected, the subjective notes section 205I may be displayed on the notes portion 815. In some examples, the subjective notes section 205I is a floating panel 2005 that is movable across the display. In this way, a second section 205A-N may be opened at the same time as the subjective notes section 205I. The floating panel 2005 may include a selectable text box 2010 that can receive one or more subjective/informal notes from a user. As such, the user is able to refer to their informal notes in the floating panel 2005 while editing one of the other section(s) 205A-N.

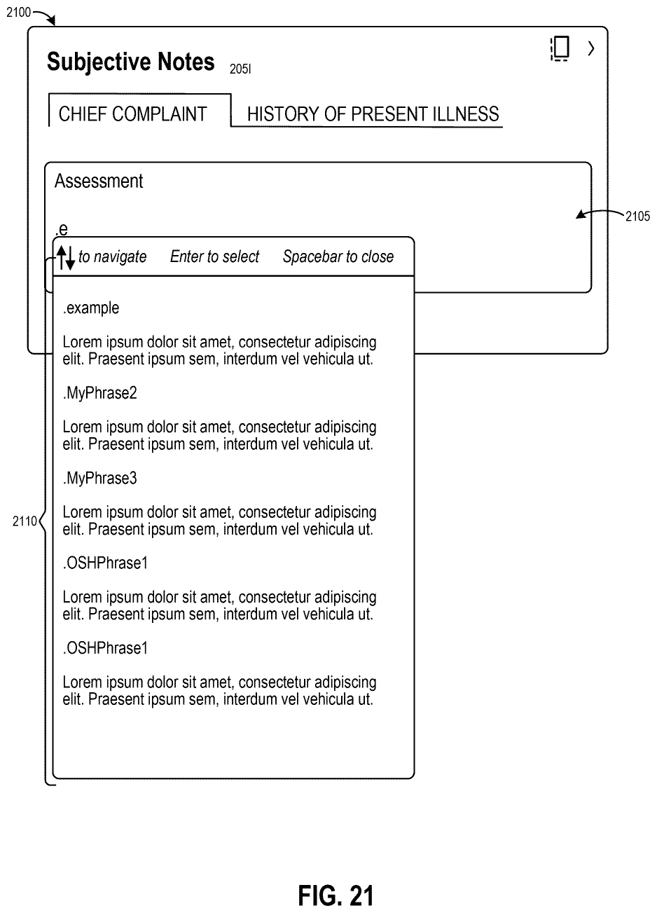

FIG. 21 depicts a user interface 2100 to illustrate the subjective notes section 205I, according to some examples. The user interface 2100 may include one or more selectable elements, such as a text box 2105 to receive notes from the user that is editing the subjective notes section 205I. In some examples, the user interface 2100 may display a list of shortcuts 2110. Each of the shortcuts 2110 include a shortcut phrase and a detailed note. When a shortcut phrase is received in the text box 2105, the text box 2105 may be populated with the detailed description. The shortcuts 2110 may include common text entries that allow the user to simply select the text instead of manually entering it.

FIG. 22 depicts a user interface 2200 to illustrate an indication that a section is locked to editing, according to some examples. The user interface 2200 shows the subjective notes section 205I in greater detail. For example, the subjective notes section 205I may include a chief complaint portion 2205 and a history of present illness section 2210. When the subjective notes section 205I is being edited by a user, indications 2215 and 2220 may be displayed. The indication 2215 may be an icon (e.g., a crossed-out pencil icon) to indicate to a user that they are unable to edit the section 205I. The indication 2220 may be a text box to indicate an identity of the user editing the section 205I. For example, the indication 2220 may read “Currently being edited by Nurse 1” when a user interacts with the section 205I.

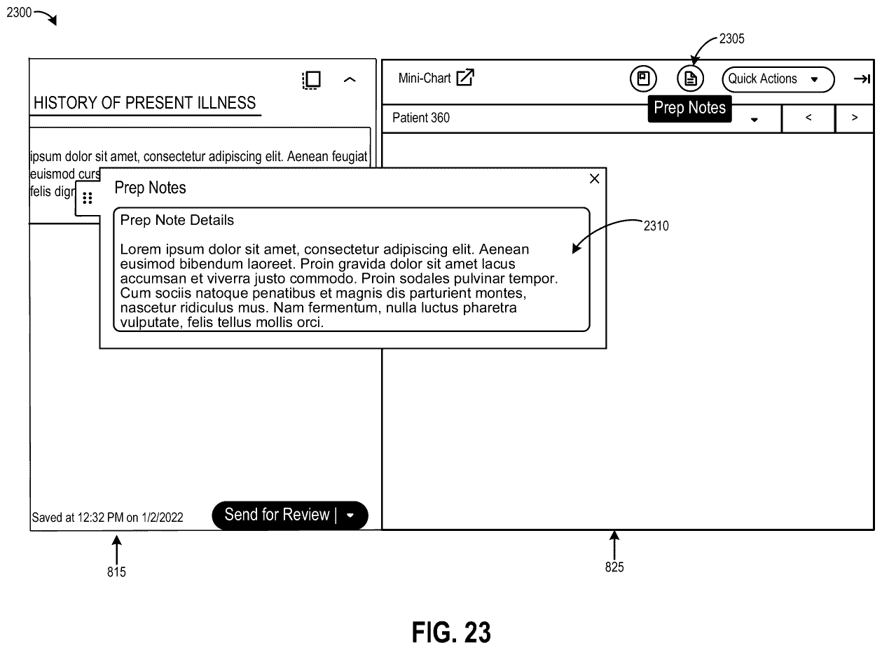

FIG. 23 depicts a user interface 2300 to illustrate a notes feature of the condensed health record 825. On a top area of the condensed health record 825, the user interface 2300 may include a selectable element 2305. When selected, the user interface 2300 displays a text box 2310 to receive notes. The text box 2310 may be movable across the user interface 2300 such that the text box 2310 can receive notes while a user is editing another section being displayed in the notes portion 815. In some examples, the text box 2310 is the floating panel 2005 of the subjective notes section 205I. In other examples, the text box 2310 is a separate text box to store other notes. For example, the floating panel 2005 may receive notes taken during an examination of the patient and the text box 2310 may receive prep notes taken before the examination occurs.

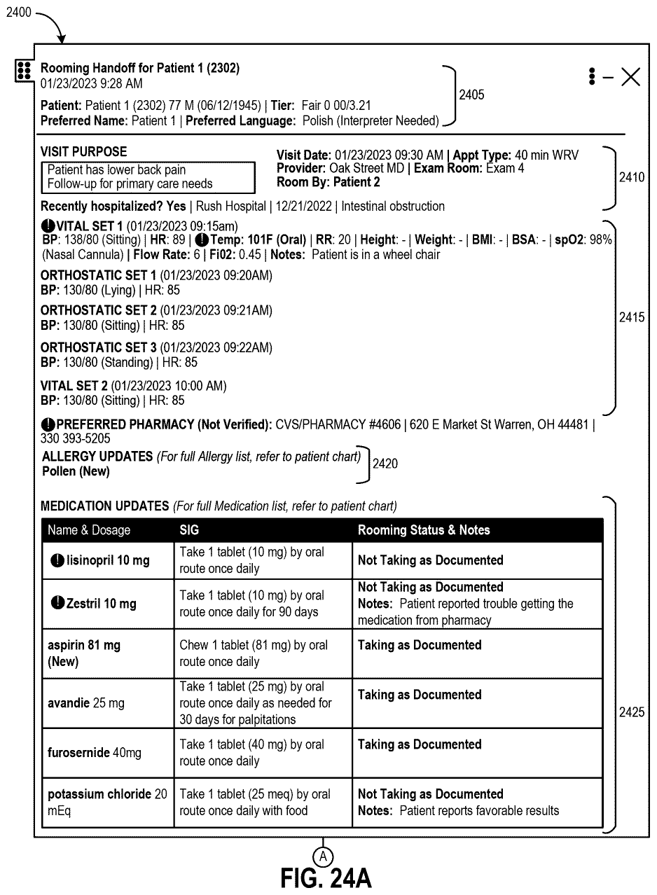

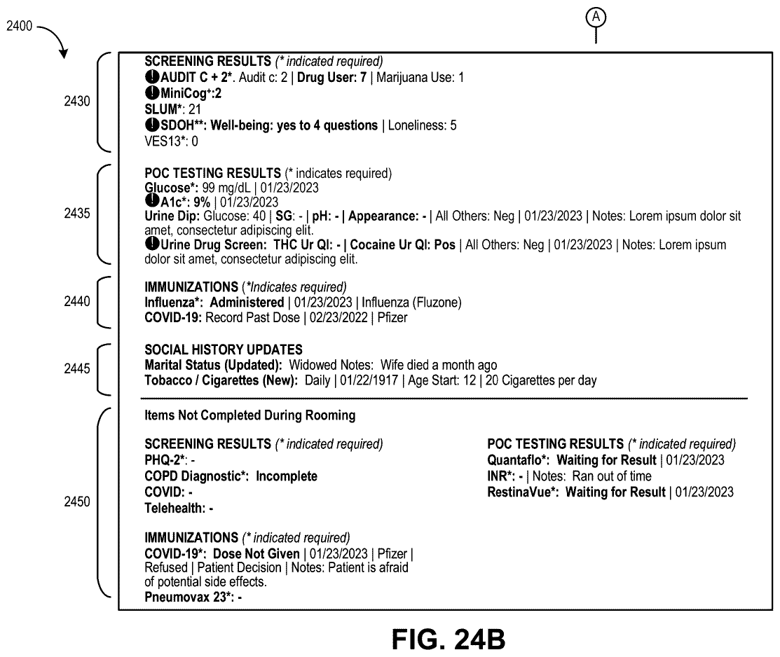

FIG. 24A depicts a user interface 2400 for a rooming handoff for a patient, according to some examples. In some examples, the user interface 2400 may display a document to summarize all of the patient records recorded during the rooming process. The user interface 2400 may be generated as a result of a handoff element, such as the handoff element 820, being selected by a user. A handoff may refer to a process of transferring the patient's rooming information to the user(s), who will be performing the examination process, such that the rooming notes may be referenced during the examination. The user interface 2400 may include summarized patient information 2405, such as a patient's name, age, preferred language, time of visit, etc. The user interface 2400 may include a visit purpose summary 2410. The visit purpose summary 2410 may include a portion of the information recorded in the visit purpose section 205A. For example, the visit purpose summary 2410 may include primary visit purpose notes and notes regarding recent hospitalization. The user interface 2400 may include a vitals summary 2415, which may include a portion of the information recorded in the vitals section 205B. For example, the vitals summary 2415 may display a completed main vital set and a time that the vitals were completed. The user interface 2400 may also include an allergy summary 2420, a medication summary 2425, a screening summary 2430, a lab test summary 2435, an immunization summary 2440, and a social history 2445, all of which include portions of the records entered to any of the sections A-N.

FIG. 24B continues to depict the user interface 2400, according to some examples. The user interface 2400 includes a portion 2450. The portion 2450 may display items that were not completed/notes that were not entered during the rooming process. For example, the portion 2450 may display a message to indicate that the patient has not been given a dose of a required vaccination. As another example, the portion 2450 may display a message to indicate that the patient has not performed one of their required screenings.

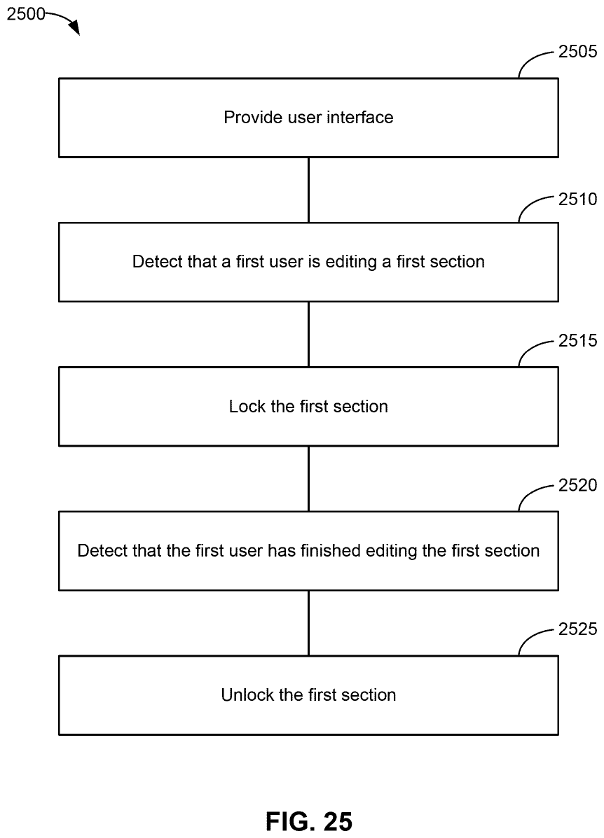

FIG. 25 depicts a flow chart of a method 2500, according to some examples. In some examples, the method 2500 may be implemented and/or executed during the process 200. In some examples, the system 100 and/or one or more systems, components, elements, and/or devices thereof may implement the method 2500 and/or one or more steps thereof. For example, the processing circuits 110 may implement at least one step of the method 2500. In some examples, the method 2500 and/or one or more steps thereof may be modified and/or changed such that steps may be added, removed, combined, separated, repeated, omitted, skipped, reproduced, replicated, and/or otherwise altered. For example, a first step may be combined with a second step. As another example, a first step may be split into two or more discrete and/or separate steps.

In some examples, at step 2505, a user interface may be provided. For example, the interface generator 130, or any other component of the system 100, may provide a user interface comprising a plurality of sections 205A-N. The sections 205A-N can capture a plurality of different pieces of information regarding at least one of a patient or a meeting.

In some examples, at step 2510, a detection that a first user is editing a first section may occur. For example, the locking circuit 135, or any other component of the system 100, may detect that a first user of the plurality of users (e.g., the user of user device 160A) is editing a first section of the plurality of sections (e.g., section 205A).

In some examples, at step 2515, the first section may be locked. For example, the locking circuit 135, or any other component of the system 100, may lock the section 205A to editing by a plurality of other users of the plurality of users. For example, the section 205A may be locked to the users of the user devices 160B-N. The section 205A may be locked responsive to detecting that the user of the user device 160A is editing the section 205A.

In some examples, at step 2520, a detection that the first user has finished editing the first section may occur. For example, the locking circuit 135, or any other component of the system 100, may detect that the user of the user device 160A has finished editing the section 205A.

In some examples, at step 2525, the first section may be unlocked. For example, the locking circuit 135, or any other component of the system 100, may unlock the section 205A to editing by any of the users of the user devices 160A-N. The locking circuit 135 may unlock the section 205A responsive to detecting that the user of the user device 160A has finished editing the section 205A. In some examples, the first section may be unlocked, responsive to detecting that the user device 160A has been inactive for a predetermined time threshold. For example, the locking circuit 135 may unlock the section 205A if there are no edits received for a 10-minute period. The predetermined time threshold can vary in duration, for example the threshold may be 10 minutes, five minutes, three minutes, etc.

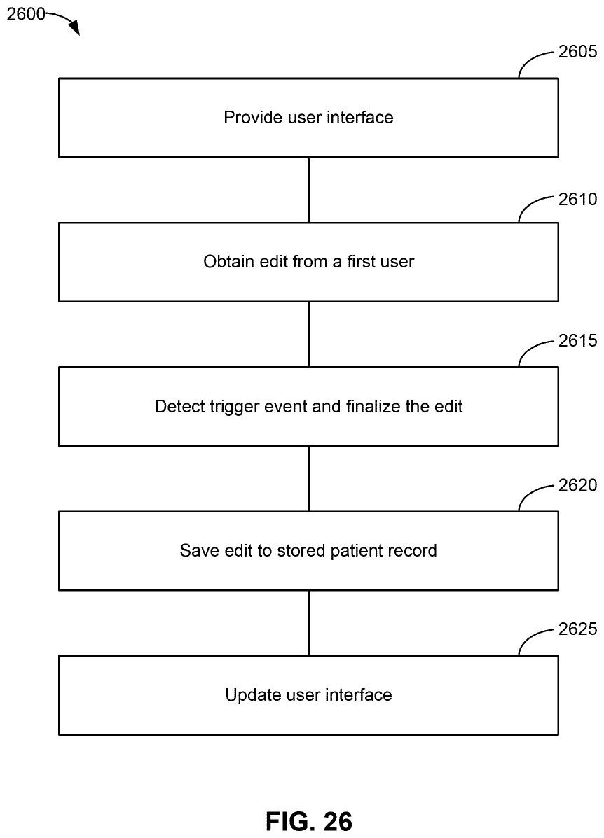

FIG. 26 depicts a flow chart of method 2600, according to some examples. In some examples, the method 2600 may be implemented and/or executed during the process 200. In some examples, the system 100 and/or one or more systems, components, elements, and/or devices thereof may implement the method 2600 and/or one or more steps thereof. For example, the processing circuits 110 may implement at least one step of the method 2600. In some examples, the method 2600 may be added, removed, combined, separated, repeated, omitted, skipped, reproduced, replicated, and/or otherwise altered. For example, a first step may be combined with a second step. As another example, a first step may be split into two or more discrete and/or separate steps.

In some examples, at step 2605, a user interface may be provided. For example, the processing circuit 110 may provide a first user interface capturing information regarding at least one of the patient or the visit. In some examples, the first user interface may be provided by the interface generator 130. In some examples, the information may include a plurality of sections 205A-N. For example, the information may include a vitals section 205B, a medication section 205D, and an allergy section 205G, among others.

In some examples, at step 2610, an edit may be obtained from a first user. For example, the edit to the information may be obtained, via the first user interface, from the first user. In some examples, the first user interface may include a plurality of selectable elements (e.g., buttons, drop-down menu selections, text boxes, etc.) that are configured to receive the edits from the first user.

In some examples, at step 2615, a trigger event may be detected, and the edit may be finalized. For example, the processing circuit 110 may detect the trigger event. The trigger event may be at least one of an information approval event, received from either an input on the user interface or as a result of the edits being processed by the processing circuit 110. Responsive to detecting the trigger event, the processing circuit 110 may finalize the edit to the information from the first user. For example, the data circuit 125, or another component in the data processing system, may validate the edits such that they can be used to update the stored record.

In some examples, at step 2620, the edit to the stored record may be saved. For example, responsive to finalizing the edit, the processing circuit 110 may save the edit to the stored record for the patient. The edit may be stored in at least one of the database 140 or database 157 in a designated area.

In some examples, at step 2625, the user interface is updated. For example, responsive to finalizing the edit, the processing circuit 110 may push the update based on the edit to at least one of the first user interface being viewed by the first user or a second user interface being viewed by a second user. Pushing the update causes a portion of at least one of the first user interface or the second user interface associated with the stored record to be updated, based on the edit.

The arrangement, construction, and description of the systems and methods as shown in the various exemplary examples are illustrative only. While some examples have been described herein, several modifications and/or adjustments are possible (e.g., variations in sizes, dimensions, structures, shapes, and proportions of the various elements, values of parameters, mounting arrangements, use of materials, colors, orientations, etc.). For example, the position of elements can be reversed or otherwise varied, and the nature or number of discrete elements or positions can be altered or varied. Accordingly, all such modifications are intended to be included within the scope of the present disclosure. The order or sequence of any process or method steps can be varied or re-sequenced according to alternative examples. Other substitutions, modifications, changes, and omissions can be made in the design, operating conditions, and arrangement of the exemplary examples without departing from the scope of the present disclosure.

The present disclosure contemplates methods, systems, and program products on any machine-readable media for accomplishing various operations. The examples of the present disclosure can be implemented using existing computer processors, or by a special purpose computer processor for an appropriate system, incorporated for this or another purpose, or by a hardwired system. Examples within the scope of the present disclosure include program products comprising machine-readable media for carrying or having machine-executable instructions or data structures stored thereon. Such machine-readable media can be any available media that can be accessed by a general purpose or special purpose computer or other machine with a processor. By way of example, such machine-readable media can comprise RAM, ROM, EPROM, EEPROM, CD-ROM or other optical disk storage, magnetic disk storage or other magnetic storage devices, or any other medium, which can be used to carry or store desired program code in the form of machine-executable instructions or data structures, and which can be accessed by a general purpose or special purpose computer or other machine with a processor. Combinations of the above are also included within the scope of machine-readable media. Machine-executable instructions include, for example, instructions and data, which cause a general-purpose computer, special purpose computer, or special purpose processing machines to perform a certain function or group of functions.

As used herein, an element or step recited in the singular and proceeded with the word “a” or “an” should be understood as not excluding plural elements or steps, unless such exclusion is explicitly recited. Furthermore, references to “exemplary example,” “one example,” or “some example” of the present disclosure are not intended to be interpreted as excluding the existence of additional examples that also incorporate the recited features.

It should be noted that the term “exemplary” and variations thereof, as used herein to describe various examples, are intended to indicate that such examples are possible examples, representations, or illustrations of possible examples (and such terms are not intended to connote that such examples are necessarily extraordinary or superlative examples).

The patent claims at the end of this document are not intended to be construed under 35 U.S.C. § 112(f), unless traditional means-plus-function language is expressly recited, such as “means for” or “step for” language being expressly recited in the claim(s).

Although the figures show a specific order of method steps, the order of the steps may differ from what is depicted. Also, two or more steps can be performed concurrently or with partial concurrence. Such variation will depend on the software and hardware systems chosen and on designer choice. All such variations are within the scope of the disclosure. Likewise, software implementations could be accomplished with standard programming techniques with rule-based logic and other logic to accomplish the various connection steps, processing steps, comparison steps and decision steps.

Conditional language used herein, such as, among others, “can,” “could,” “might,” “may,” “e.g.,” and the like, unless specifically stated otherwise, or otherwise understood within the context as used, is generally intended to convey that certain examples include, while other examples do not include, certain features, elements and/or steps. Thus, such conditional language is not generally intended to imply that features, elements and/or steps are in any way required for one or more examples, or that one or more examples necessarily include logic for deciding, with or without other input or prompting, whether these features, elements and/or steps are included or are to be performed in any particular example. The terms “comprising,” “including,” “having,” and the like are synonymous and are used inclusively, in an open-ended fashion, and do not exclude additional elements, features, acts, operations, and so forth. Also, the term “or” is used in its inclusive sense (and not in its exclusive sense) so that when used, for example, to connect a list of elements, the term “or” means one, some, or all of the elements in the list.

It may be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It may be further understood by those within the art that if a specific number of an introduced claim recitation is intended, such an intent may be explicitly recited in the claim, and in the absence of such recitation, no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to inventions containing only one such recitation, even when the same claim includes the introductory phrases “one or more” or “at least one” and indefinite articles such as “a” or “an” (e.g., “a” and/or “an” should typically be interpreted to mean “at least one” or “one or more”); the same holds true for the use of definite articles used to introduce claim recitations. In addition, even if a specific number of an introduced claim recitation is explicitly recited, those skilled in the art may recognize that such recitation should typically be interpreted to mean at least the recited number (e.g., the bare recitation of “two recitations,” without other modifiers, typically means at least two recitations, or two or more recitations). Furthermore, in those instances where a convention analogous to “at least one of A, B, and C” is used, or “at least one of A, B, or C” is used, in both cases such usage should be interpreted as covering any permutation of A, B, or C, alone or in combination, unless expressly indicated otherwise (e.g., “a system having at least one of A, B, and C” would include systems that have A alone, B alone, C alone, A and B together, A and C together, B and C together, and A, B, and C together). It may be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” may be understood to include the possibilities of “A” or “B” or “A and B.” Further, unless otherwise noted, the use of the words “approximate,” “about,” “around,” “substantially,” etc., mean plus or minus ten percent.

The term “coupled” and variations thereof, as used herein, means the joining of two members, directly or indirectly, to one another. Such joining may be stationary (e.g., permanent, or fixed) or moveable (e.g., removable, or releasable). Such joining may be achieved with the two members coupled directly to each other, with the two members coupled to each other using a separate intervening member and any additional intermediate members coupled with one another, or with the two members coupled to each other using an intervening member that is integrally formed as a single unitary body with one of the two members. If “coupled” or variations thereof are modified by an additional term (e.g., directly coupled), the generic definition of “coupled” provided above is modified by the plain language meaning of the additional term (e.g., “directly coupled” means the joining of two members without any separate intervening member), resulting in a narrower definition than the generic definition of “coupled” provided above. Such coupling may be mechanical, electrical, or fluidic.

In various implementations, the steps and operations described herein may be performed on one processor or in a combination of two or more processors. For example, in some implementations, the various operations could be performed in a central server or set of central servers configured to receive data from one or more devices (e.g., edge computing devices/controllers) and perform the operations. In some implementations, the operations may be performed by one or more local controllers or computing devices (e.g., edge devices), such as controllers dedicated to and/or located within a particular industrial environment or portion of an industrial environment. In some implementations, the operations may be performed by a combination of one or more central or offsite computing devices/servers and one or more local controllers/computing devices. All such implementations are contemplated within the scope of the present disclosure. Further, unless otherwise indicated, when the present disclosure refers to one or more computer-readable storage media and/or one or more controllers, such computer-readable storage media and/or one or more controllers may be implemented as one or more central servers, one or more local controllers or computing devices (e.g., edge devices), any combination thereof, or any other combination of storage media and/or controllers regardless of the location of such devices.

What is claimed is:

1. A method for synchronizing notes for a visit of a patient with a healthcare provider with a stored record for the patient, the method comprising:
providing, by one or more processing circuits, a first user interface capturing information regarding at least one of the patient or the visit;
obtaining, via the first user interface, an edit to the information from a first user;
detecting a trigger event and, responsive to detecting the trigger event, finalizing, via the one or more processing circuits, the edit to the information from the first user;
responsive to finalizing the edit, saving, by the one or more processing circuits, the edit to the stored record for the patient; and
responsive to finalizing the edit, pushing, by the one or more processing circuits, an update based on the edit to at least one of the first user interface being viewed by the first user or a second user interface being viewed by a second user, wherein pushing the update comprises causing a portion of at least one of the first user interface or the second user interface associated with the stored record to be updated based on the edit.

2. The method of claim 1, wherein at least one of the first user interface or the second user interface comprises a plurality of sections capturing a plurality of different pieces of information regarding at least one of the patient or the visit, wherein the plurality of sections include at least one of: a visit purpose section, a vitals section, a screenings section, a medications section, a point of care section, an immunizations section, a history section, an allergy section, a demographics section, an assessment section, a care plan section, a physical exam section, or a subjective notes section.

3. The method of claim 1, wherein at least one of the first user interface or the second user interface includes a first portion and a second portion provided within a same display screen, comprising:
receiving the edit to the information, by the one or more processing circuits, in the first portion of the first user interface; and
pushing the update based on the edit to the second portion of the first user interface.

4. The method of claim 3, wherein the second portion is a condensed health record of the patient, wherein pushing the update based on the edit causes at least a portion of the condensed health record of the first user interface and the second user interface to update based on the edit.

5. The method of claim 1, wherein pushing the update is done within a threshold time after the edit to the information is received.

6. The method of claim 1, wherein the edit to the information is obtained from the first user interface of the first user and the update is pushed to the second user interface of the second user.

7. The method of claim 1, wherein the trigger event includes at least one of an information approval event or an editing timeout event.

8. The method of claim 1, wherein at least one of the first user interface or the second user interface comprises a plurality of sections capturing a plurality of different pieces of information regarding at least one of the patient or the visit, comprising:
detecting, using the one or more processing circuits, that the first user is editing a first section of the plurality of sections; and
responsive to detecting that the first user is editing the first section, locking, using the one or more processing circuits, the first section to editing by other users of a plurality of users.

9. The method of claim 8, comprising:
detecting, using the one or more processing circuits, that the first user has finished editing the first section; and
responsive to detecting that the first user has finished editing the first section, unlocking, using the one or more processing circuits, the first section for editing by the second user.

10. The method of claim 1, wherein at least one of the first user interface or the second user interface comprises a plurality of sections capturing a plurality of different pieces of information regarding at least one of the patient or the visit, comprising:
obtaining, via the first user interface, the edit to the information from the first user editing a first section of the plurality of sections; and
updating, via the first user interface, other sections of the plurality of sections with at least a portion of the edit to the information from the first user.

11. The method of claim 1, comprising:
capturing, via the one or more processing circuits, the edit to the information;
storing, via a database, the edit to the information; and
responsive to detecting the trigger event, saving the edit to the stored record for the patient.

12. The method of claim 1, wherein at least one of the first user interface or the second user interface comprises a plurality of sections capturing a plurality of different pieces of information regarding at least one of the patient or the visit, comprising:
displaying, via the first user interface, a first portion of the plurality of sections to the first user, the first portion of the plurality of sections based on a first role assigned to the first user; and
displaying, via the second user interface, a second portion of the plurality of sections to the second user, the second portion of the plurality of sections based on a second role assigned to the second user, the second portion including different information than the first portion and the second role being different than the first role.

13. The method of claim 1, comprising:
determining, by the one or more processing circuits, a plurality of at least one of health screening recommendations or examination recommendations related to at least one of the patient or the visit; and
providing, via the one or more processing circuits, screening information comprising the plurality of the at least one of the health screening recommendations or the examination recommendations on the first user interface.

14. The method of claim 1, comprising:
providing, via the one or more processing circuits, statement information on the first user interface, the statement information having a plurality of statement codes associated with at least one of the patient or the visit, wherein the plurality of statement codes are determined, by the one or more processing circuits, based on the information.

15. A system to synchronize notes for a visit of a patient with a healthcare provider with a stored record for the patient, the system comprising:
one or more processing circuits configured to:
provide a first user interface capturing information regarding at least one of the patient or the visit;
obtain an edit to the information from a first user;
detect a trigger event and, responsive to detecting the trigger event, finalize the edit to the information from the first user;
responsive to finalizing the edit, save the edit to the stored record for the patient; and
responsive to finalizing the edit, push an update based on the edit to at least one of the first user interface being viewed by the first user or a second user interface being viewed by a second user, wherein pushing the update comprises causing a portion of at least one of the first user interface or the second user interface associated with the stored record to be updated based on the edit.

16. The system of claim 15, wherein at least one of the first user interface or the second user interface comprises a plurality of sections capturing a plurality of different pieces of information regarding at least one of the patient or the visit, comprising the one or more processing circuits to:
detect that the first user is editing a first section of the plurality of sections; and
responsive to detecting that the first user is editing the first section, lock the first section to editing by other users of a plurality of users.

17. The system of claim 15, wherein at least one of the first user interface or the second user interface comprises a plurality of sections capturing a plurality of different pieces of information regarding at least one of the patient or the visit, wherein the plurality of sections include at least one of: a visit purpose section, a vitals section, a screenings section, a medications section, a point of care section, an immunizations section, a history section, an allergy section, a demographics section, an assessment section, a care plan section, a physical exam section, or a subjective notes section.

18. The system of claim 15, where the one or more processing circuits are further configured to:
capture the edit to the information;
store, via a database, the edit to the information; and
responsive to detecting the trigger event, save the edit to the stored record for the patient.

19. The system of claim 15, wherein at least one of the first user interface or the second user interface includes a first portion and a second portion within a same display screen, where the one or more processing circuits are further configured to:
receive the edit to the information in the first portion of the first user interface; and
push the update based on the edit to the information to the second portion of the first user interface, wherein the second portion is a summarized health record of the patient.

20. One or more non-transitory computer-readable storage media having instructions stored thereon that, when executed by one or more processors, cause the one or more processors to perform operations comprising:
providing a first user interface capturing information regarding a visit of a patient with a healthcare provider;
obtaining an edit to the information from a first user;
detecting a trigger event and, responsive to detecting the trigger event, finalizing the edit to the information from the first user;
responsive to finalizing the edit, saving the edit to a stored record for the patient; and
responsive to finalizing the edit, pushing an update based on the edit to at least one of the first user interface being viewed by the first user or a second user interface being viewed by a second user, wherein pushing the update comprises causing a portion of at least one of the first user interface or the second user interface associated with the stored record to be updated based on the edit.
